(19) United States
(12) Patent Application Publication
(10) Pub. No.: US 2008/0063637 A1
(43) Pub. Date: Mar. 13, 2008

(54) Regulation of oncogenesis by Akt-specific isoforms

(75) Inventors: Philip N. Tsichlis, Winchester, MA (US); Ioanna Maroulakou, Boston, MA (US)

Correspondence Address: MEDLEN & CARROLL, LLP, Suite 350, 101 Howard Street, San Francisco, CA, 94105, US

(73) Assignee: The Trustees of Tufts College

(21) Appl. No.: 11/804,255

(22) Filed: May 17, 2007

Related U.S. Application Data
(60) Provisional application No. 60/801,746, filed on May 19, 2006.

(51) Int. Cl. A61K 38/45; A01K 67/027; A61K 39/395; A61P 35/00

(52) U.S. Cl. 424/130.1; 424/94.5; 800/18

(57) ABSTRACT

Expression of the polyoma middle T antigen (PyMT) or the HER2/Neu oncogene in the mammary gland of transgenic mice from MMTV LTR-driven expression constructs, gives rise to mammary adenocarcinomas. Here we show that ablation of Akt1 inhibits, while ablation of Akt2 accelerates tumor induction by both transgenes. The tumors arising in mice lacking individual Akt isoforms exhibit distinct histologic phenotypes. Although all these tumors are locally invasive however, they differ in metastatic potential, with the Akt1^{-/-} tumors being less metastatic. The development of the mammary gland during puberty and the expression of MMTV LTR-driven transgenes in Akt1, Akt2 and Akt3 knockout mice are normal. These data combined, indicate that ablation of individual Akt isoforms influences tumor induction by modulating oncogenic signaling induced by the two transgenes. Further studies revealed that the delay of tumor induction in Akt1 knockout mice is due to the inhibitory affects of Akt1 ablation on cell proliferation and cell survival. The results of the present study may have significant translational implications because they may influence how Akt inhibitors will be used in the treatment of human cancer.

### FIELD OF THE INVENTION

The present invention is related to the development of anti-cancer agents. In one embodiment, the anti-cancer agent regulates the PI3K/Akt pathway. In one embodiment, the anti-cancer agent comprises an Akt protein. In one embodiment, the anti-cancer agent comprises an Akt2 protein. In one embodiment, the anti-cancer agent comprises an Akt1 inhibitor.

### BACKGROUND

During cancer development, tumor cells acquire a number of phenotypic characteristics that allow them to proliferate both rapidly and limitlessly, invade the surrounding tissue, survive without their normal microenvironment, and finally, metastasize to secondary sites. Hanahan et al., Cell 100:57-70 (2000). These features are usually acquired progressively over a protracted period of time as a result of increasing genomic instability that leads to up-regulation of oncogenes and down-regulation of tumor suppressor genes. As a result of intensive worldwide research activity in the last few years, the major signaling pathways that are altered during tumorigenesis, and how these pathways link to dysregulated processes such as proliferation and survival, are being elucidated.

The serine/threonine protein kinase, protein kinase B or Akt (PKB/Akt), has been identified as a potential regulator of widely divergent cellular processes including apoptosis, proliferation, differentiation, and metabolism. Nicholson et al., “The protein kinase B/Akt signaling pathway in human malignancy” Cellular Signaling 14: 381-395 (2002). Disruption of normal PKB/Akt signaling has now been documented as a frequent occurrence in several human cancers and the enzyme appears to play a role in their progression. Dysregulation of PKB/Akt signaling mechanisms could, therefore, contribute to cancer progression. Several recent reviews contain detailed descriptions of PKB/Akt structure and its molecular mechanism of activation. Kandel et al., Exp Cell Res 253:210-29 (1999); Datta et al., Genes Dev 13:2905-27 (1999); and Vanhaesebroeck et al., Biochem J 346:561-76 (2000).

Breast cancer in humans arises as a result of transformation of various cell lineages. However, the great majority of breast cancers are epithelial in origin. Human breast cancer is classified into various histologic subtypes, based on the origin and the state of differentiation of the transformed cells. The most common are ductal adenocarcinomas, which account for about 85% of all the cases. Clinical prognosis of breast cancer is based upon histological alterations. Thus, agents that prevent histological alterations in the mammary ductal architecture would be expected to have anti-cancer efficacy.

What is needed is an anti-cancer agent that increases tumoral apoptosis, decreases tumoral proliferation and has no effect on non-cancerous intracellular cell structure.

### SUMMARY

The present invention is related to the development of anti-cancer agents. In one embodiment, the anti-cancer agent regulates the PI3K/Akt pathway. In one embodiment, the anti-cancer agent comprises an Akt protein. In one embodiment, the anti-cancer agent comprises an Akt2 protein. In one embodiment, the anti-cancer agent comprises an Akt1 inhibitor.

In one embodiment, the present invention contemplates a method, comprising: a) providing; i) a patient, said patient comprises a tumor; ii) a composition comprising an Akt2 isoform protein; and b) administering said composition to said patient under conditions such that said tumor becomes reduced in size. In one embodiment, the tumor comprises a mammary tumor. In one embodiment, the tumor comprises a thymic tumor. In one embodiment, the protein is a fusion protein. In one embodiment, the administering is intratumoral. In one embodiment, the administering is parenteral.

In one embodiment, the present invention contemplates a method, comprising: a) providing; i) a patient, said patient comprises a tumor; ii) a composition comprising an Akt1 isoform protein inhibitor; and b) administering said composition to said patient under conditions such that said tumor becomes reduced in size. In one embodiment, the tumor comprises a mammary tumor. In one embodiment, the tumor comprises a thymic tumor. In one embodiment, the inhibitor is selected from the group consisting of a small molecule and an antibody. In one embodiment, the administering is intratumoral. In one embodiment, the administering is parenteral.

In one embodiment, the present invention contemplates a transgenic mouse, comprising an MMTV-PyMT transgene operably linked to an Akt1^{-/-} genotype. In one embodiment, the mouse further comprises a developing tumor. In one embodiment, the tumor development is inhibited relative to an MMTV-PyMT Akt^{+/+} transgenic mouse. In one embodiment, the mouse comprises histologically normal tissue selected from the group consisting of mammary ductal architecture and mammary epithelial proliferation.

In one embodiment, the present invention contemplates a transgenic mouse, comprising an MMTV-HER2/Neu transgene operably linked to an Akt1^{-/-} genotype. In one embodiment, the mouse further comprises a developing tumor. In one embodiment, the tumor development is inhibited relative to an MMTV-PyMT Akt^{+/+} transgenic mouse. In one embodiment, the mouse comprises histologically normal tissue selected from the group consisting of mammary ductal architecture and mammary epithelial proliferation.

In one embodiment, the present invention contemplates an MMTV-PyMT transgene operably linked to an Akt2^{-/-} genotype. In one embodiment, the mouse further comprises a developing tumor. In one embodiment, the tumor development is accelerated relative to an MMTV-PyMT Akt^{+/+} transgenic mouse. In one embodiment, the mouse comprises histologically normal tissue selected from the group consisting of mammary ductal architecture and mammary epithelial proliferation.

In one embodiment, the present invention contemplates a transgenic mouse, comprising an MMTV-HER2/Neu transgene operably linked to an Akt2^{-/-} genotype. In one embodiment, the mouse further comprises a developing tumor. In one embodiment, the tumor development is accelerated relative to an MMTV-PyMT Akt^{+/+} transgenic mouse. In one embodiment, the mouse comprises histologically normal tissue selected from the group consisting of mammary ductal architecture and mammary epithelial proliferation.

### DEFINITIONS

The term “tumor” as used herein, refers to an abnormal benign or malignant new growth of tissue that possesses no physiological function and arises from uncontrolled usually rapid cellular proliferation.

The term “reduced in size” means any quantitative reduction of a three dimensional measurement (i.e., for example, circumference, length, width, height, density etc.) of a physical object (i.e., for example, tumor, epithelial layer etc.).

“Vector” shall be defined as a circular double-strand DNA molecule capable of having any genes therein encoded transcribed when put into the appropriate environment in vivo or in vitro.

“Expression” shall be defined as the transcription and translation of a gene. Such transcription and translation may be in vivo or in vitro.

“Constitutive” shall be defined as the level of expression of a genomic gene in vivo.

“Overexpression” shall be defined as expression at a level above the level normally expressed by an untransfected cell and is reflected by the combined expression level of a genomic gene along with a similar gene transfected into a cell.

“Transfect” shall be defined as the introduction of a vector into a cell by means such as, e.g., electroporation of lipofectamine.

“In operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “compound” as used herein refers to organic or inorganic molecules. The term includes, but is not limited to polypeptides, proteins, glycoproteins (e.g. antibodies), nucleic acids, oligonucleotides, and inorganic molecules.

The term “small molecule”, as used herein, refers to a compound that is an organic molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Daltons.

The term “protein” herein is meant at least two covalently attached amino acids, which includes proteins, polypeptides, oligopeptides, and peptides. The protein may be made of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline, and norleucine are considered amino acids for the purposes of this invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger and Mungall (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141), phosphorothioate (Mag et al. (1991) Nucleic Acids Res. 19:1437; and U.S. Pat. No. 5,644,048), phosphorodithioate (Brill et al. (1989) J. Am. Chem. Soc. 111:2321), O-methyiphophoroamidite linkages (see Eckstein (1991) Oligonucleotides and Analogues: A Practical Approach, Oxford University Press), and peptide nucleic acid backbones and linkages (see Egholm et al. (1992) J. Am. Chem. Soc. 114:1895; Meier et al. (1992) Chem. Int. Ed. Engl. 31: 1008; Egholm et al. (1993) Nature, 365: 566; Carlsson el al. (1996) Nature 380: 207). Other analog nucleic acids include those with positive backbones (Dempcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; von Kriedrowski et al. (1991) Agnew. Chem. Intl. Ed. English 30: 423; Letsinger et al. (1988) J. Am. Chem. Soc. 110:4470; Jung et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook (1994); De Mesmaeker and Wardner (1994), Bioorganic & Medicinal Chem. Lett. 4: 395; Gao and Jeffs (1994) J. Biomolecular NMR 444:17; Hon et al. (1996) Tetrahedron Lett. 37:743) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook (1994). Nucleic acids containing one or more carbocyclic sugars are also included within the definition of nucleic acids (see Jenkins and Tuner (1995), Chem. Soc. Rev. 24:169-176. Several nucleic acid analogs are described in Rawls, C & E News Jun. 2, 1997 page 75:35 These modifications of the ribose-phosphate backbone may be done to facilitate the addition of additional moieties such as labels, or to increase the stability and half-life of such molecules in physiological environments.

“In vivo” refers to in the living body of an organism.

“In vitro” refers to outside the living body, such as, an artificial environment, for example, a test tube or a cell or tissue culture.

The term “accelerates” as used herein, refers to an interaction between a molecule and a biological pathway or system, that causes a change (e.g., enhancement) in the biological pathway or system. Molecules resulting in acceleration may include proteins, nucleic acids, carbohydrates, or any other molecules which bind or interact with biologically active molecules. For example, agonists can alter the activity of gene transcription by interacting with RNA polymerase directly or through a transcription factor.

The term “inhibited,” as used herein, refer to a molecule which, when interacting with a biologically active molecule, blocks or modulates the biological activity of the biologically active molecule. Antagonists and inhibitors may include proteins, nucleic acids, carbohydrates, or any other molecules that bind or interact with biologically active molecules. Inhibitors and antagonists can effect the biology of entire cells, organs, or organisms (e.g., an inhibitor that slows tumor growth).

The term “transgenic”, as used herein, refers to any host (plant, animal, or microbial) being, or used to produce, an organism or cell of one species into which one or more genes of other species have been incorporated. For example, a transgenic mouse may comprise an Akt knockout gene and/or an oncogenic transgene.

The term “antibody”, as used herein, refers to any protein of a high molecular weight that are produced normally by specialized B cells after stimulation by an antigen and act specifically against the antigen in an immune response, that are produced abnormally by some cancer cells, and that typically consist of four subunits including two heavy chains and two light chains.

The term “accessible”, as used herein, refers to any ability to treat a solid tumor by non-surgical techniques. Such techniques may include, but are not limited to, injection into the skin or injection via endoscopy, bronchoscopy, cystoscopy, colonoscopy, laparoscopy, catheterization, or topical application by a lotion, ointment or powder. For example, an ovarian solid tumor may be accessible by laparoscopy. In another example, a colon solid tumor may be accessible by colonoscopy.

The term “introducing”, as used herein, refers to any method of transferring a compound into a tissue and subsequently into cells within said tissue. Such methods of introduction may include, but are not limited to, viral vectors, retroviral vectors, adenoviral vectors, biobalistics, lipofection, and many commercially available DNA vectors known in the art. Alternatively, a compound may be placed adjacent to a cell such that the compound is incorporated into the cell by physiological mechanisms (i.e., for example, hydrophobic interactions or active transport). One method of introduction comprises injection, wherein a compound is placed directly into the intercellular space within the injected tissue. Such an injection may be possible when an organ part, growth (i.e., for example, a solid tumor), or bodily cavity is “accessible”.

The term “into”, as used herein, refers to the successful penetration of a molecule through or within a cell membrane. For example, a molecule may be introduced into a solid tumor cell by an “intratumoral injection”.

The term “regression”, “is at least partially diminished in size” or “reduced”, as used herein, refers to a diminution of a bodily growth, such as, for example, a solid tumor. Such a diminution may be determined by a reduction in measured parameters such as, but not limited to, diameter, mass (i.e., weight), or volume. The diminution by no means indicates that the size is completely reduced, only that a measured parameter is quantitatively less than a previous determination.

The term “destruction”, as used herein, refers to the complete cellular breakdown of a bodily growth, such as, for example, a solid tumor. Such a destruction may involve intracellular apoptosis and/or macrophage phagocytosis such that the bodily growth is completely digested and eliminated from the body.

The term “growth”, as used herein, refers to any tissue or organ that comprises a cellular mass considered to represent an abnormal proliferation. Such growths may be cancerous, non-cancerous, malignant, or non-malignant. If a growth comprises cancer, it may be a tumor. Such tumors may be solid or non-solid.

The term “accessible through the skin”, as used herein, refers to any non-surgical technique that is capable of reaching an internal organ or body cavity. Such non-surgical techniques do not require conventional open site surgery comprising a scalpel incision. Non-surgical techniques include, but are not limited to, percutaneous access, or bodily orifice access to internal organs or body cavities. For example, percutaneous access may include, but is not limited to, laparoscopy and catheterization. In another example, bodily orifice access, may include, but is not limited to, endoscopy, bronchoscopy, cystoscopy, and colonoscopy.

The term “patient”, as used herein, refers to any organism that is capable of developing a tumor. Such organisms include, but are not limited to, human, dog, cat, horse, cow, sheep, goat, mouse, rat, guinea pig, monkey, avian, reptiles etc.

The term “liposome-free”, as used herein, refers to any composition, mixture, or solution that does not contain sufficient lipids (i.e., for example, phospholipids) to attain a critical micellular concentration (CMC). Lipids, in fact, may be present but not at concentrations such that liposomes may form.

The term “isolated’, as used herein, refers to any composition or mixture that has undergone a laboratory purification procedure including, but not limited to, extraction, centrifugation, chromatographic separation (i.e., for example, thin layer chromatography or high performance liquid chromatography). Usually such a purification procedures provides an isolated composition or mixture based upon physical, chemical, or electrical potential properties. Depending upon the choice of procedure an isolated composition or mixture may contain other compositions, compounds or mixtures having similar chemical properties. For example, an isolated composition or mixture may contain between 1-20%, preferably, 1-10%, but more preferably 1-5% of compositions or mixtures having similar chemical properties. In one embodiment, an isolated composition or mixture comprises a mixture of glycolipids free of cholesterol and phospholipids. In one embodiment, an isolated composition or mixture comprises glycolipids having from between 5-15 glycosidic linkages.

The term “molecule”, as used herein, refers to the smallest particle of a composition that retains all the properties of the composition and is composed of one or more atoms. These one or more atoms are arranged such that the molecule may interact (i.e., ionically, covalently, non-covalently etc) with other molecules to form attachments and/or associations. For example, a molecule may have one or more atoms arranged to provide a capability for an interation with an Akt isoform protein and/or gene.

The term “formulation” or “pharmaceutical formulation”, as used herein, refers to any composition intended for the administration of a pharmaceutical compound, or combination, including, but not limited to, any chemical or peptide, natural or synthetic, that is administered to a patient for medicinal purposes. Specifically, a formulation may comprise either a single compound or a plurality of compounds.

The term “compounded” or “compounded formulation”, as used herein, refers to any formulation containing a plurality of compounds, wherein the compounds may have the same, or different dosage ratios, and further wherein the compounds may be uniform (i.e., evenly mixed) or non-uniform (i.e., unevenly mixed, including but not limited to, separated tablet layers or separated capsule compartmentalization).

The term “tablets”, as used herein, refers to any solid formulation comprising at least one pharmaceutical compound intended for oral or intrapulmonary administration to a patient. In one embodiment, “tablets” may have multiple layers (i.e., multilayered tablets), wherein each layer comprises different pharmaceutical formulation.

The term “capsules”, as used herein, refers to any polymer film-based container comprising a single or plurality of compartments containing at least one pharmaceutical compound intended for oral or intrapulmonary administration to a patient. In one embodiment, “capules” may have multiple compartments (i.e., multi-compartmentalized), wherein each compartment comprises a different pharmaceutical formulation.

The term “parenteral”, refers to the administration of a molecule, composition, or formulation that does not involve the gastrointestinal system. For example, parenteral is generally referred to in regards to an injection (i.e., for example, intravenous, intraperitoneal, intramuscular, subcutaneous, or intratumoral). A topical (i.e., for example, transdermal) route of administration is also within the scope of parenteral administration.

### BRIEF DESCRIPTION OF THE FIGURES

FIG. 1 provides exemplary short-term data showing tumor development in MMTV-HER2/Neu and MMTV-PyMT transgenic mice, wherein an Akt1 ablation protects, while an Akt2 ablation promotes the development of mammary adenocarcinomas. Panel A: Diamonds—MMTV c-neu/Akt1^{+/+}. Squares—MMTV c-neu/Akt^{-/-}. Panel B: Diamonds—MMTV c-neu (het)/Akt2^{+/+}. Squares—MMTV c-neu(het)/Akt1^{-/-}. Panel C: Diamonds—PyMT/Akt1^{+/+}. Squares—PyMT/Akt^{-/-}. Panel D: Diamonds—PyMT/Akt2^{+/+}. Squares—PyMT/Akt2^{-/-}. In the latter mice, the ablation of both Akt1 and Akt2 appear to protect from metastasis (data not shown).

FIG. 2 illustrates one embodiment of a proposed domain structure of the three putative human PKB/Akt isoforms and the PKBγ/Akt3 splice variant. Hatched bars: N-terminal PH domain.

FIG. 3 illustrates one embodiment of cell survival regulation by PKB/Akt. Black Boxes Direct PKB/Akt substrates.

FIG. 4 presents exemplary gel electrophoresis data showing that Akt1, Akt2, and Akt3 are all expressed in the mammary gland.

FIG. 5 provides exemplary long-term data showing tumor development in MMTV-HER2/Neu and MMTV-PyMT transgenic mice having the following genotypes: Panel A: Akt1^{-/-}—Diamonds^{+/+}. Squares^{-/-}. Panel B: Akt2^{-/-}—Diamonds^{+/+}. Squares^{-/-}. Akt3^{-/-}—Diamonds^{+/+}. Squares^{-/-}.

FIG. 6 provides exemplary data showing that ablation of Akt1 inhibits the metastatic potential, but not the local invasiveness, of mammary carcinomas in MMTV-PyMT transgenic mice.

FIG. 7 provides exemplary data showing that in mammary adenocarcinomas developing in either MMTV-HER2/Neu (Panel A) or MMTV-PyMT (Panel B) transgenic mice have upregulated Akt1 and downregulated Akt2.

FIG. 8 provides exemplary histological data showing the pathology of various mammary adenocarcinomas produced in MMTV-PyMT transgenic mice having the following genotypes. Panel A: Wild Type. Panel B: Akt1^{-/-}. Panel C: Akt2^{-/-}. Panel D: Akt3^{-/-}.

FIG. 9 provides exemplary histological data showing the pathology of various lung metastatic foci produced in MMTV-PyMT transgenic mice having the following genotypes. Panel A: Wild Type. Panel B: Akt1^{-/-}. Panel C: Akt2^{-/-}. Panel D: Akt3^{-/-}.

FIG. 10 provides exemplary histological data showing unaffected mammary ductal outgrowths in MMTV-PyMT transgenic mice having the following genotypes. Panel A: Wild Type. Panel B: Akt1^{-/-}. Panel C: Akt2^{-/-}. Panel D: Akt3^{-/-}.

FIG. 11 provides exemplary histological data showing unaffected branching morphology and alveolar outgrowth size in young MMTV-HER2/Neu transgenic mice having the following genotypes. Panel A: Akt1^{-/-}. Panel B: Akt1 Wild Type. Panel C: Akt2^{-/-}. Panel D: Akt2 Wild Type.

FIG. 12 provides exemplary histological data showing unaffected branching morphology and alveolar outgrowth size in young MMTV-PyMT transgenic mice having the following genotypes. Panel A: Akt1^{-/-}. Panel B: Akt1 Wild Type. Panel C: Akt2^{-/-}. Panel D: Akt2 Wild Type.

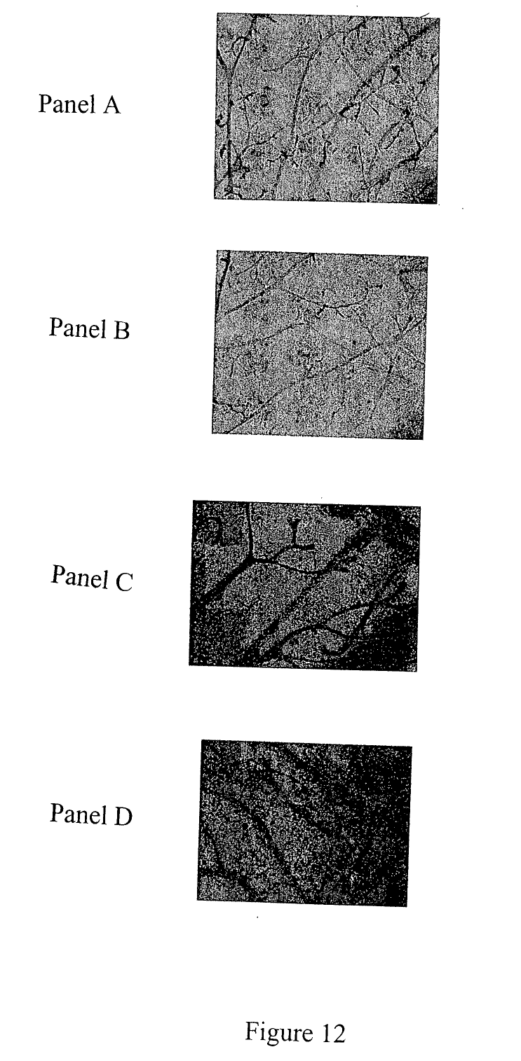

FIG. 13 provides exemplary histochemical data showing that Akt1 ablation does not inhibit HER2/Neu transgene expression. Panel A: Akt1 Wild Type Panel B: Akt1^{-/-}. Panel C: No transgene (control). Panel D: Representative gel analysis showing c-neu expression in each Akt1 genotype.

FIG. 14 provides exemplary data showing that aleveolar outgrowths grow and form hyperplastic foci significantly faster in MMTV-PyMT/Akt1^{+/+} as opposed to MMTV-PyMT/Akt^{-/-} transgenic Mice. Panel A: Eight week old mice. Panel B. Twelve week old mice. Panel C: Thirty week old mice.

FIG. 15 provides exemplary data showing that PyMT-expressing mammary epithelia from Wild Type (i.e., Akt^{+/+}) mice exhibit a more robust cell proliferation than Akt1^{-/-} epithelia. Data was collected using the stained Ki-67 proliferation marker. Panel A: Eight week old Akt1^{+/+} (hyperplasia) versus eight week old Akt1^{-/-} (early hyperplasia). Panel B: Twelve week old Akt1^{+/+} (hyperplasia) versus twenty-seven week old Akt1^{-/-} (hyperplasia). Panel C: Twelve week old Akt1^{+/+} (carcinoma) versus twenty seven week old Akt1^{-/-} (carcinoma). Panel D: Twelve week old Akt^{+/+} (metastatic lesion).

FIG. 16 provides exemplary summary data showing the percentage of stained Ki-67 marker cells in accordance with FIG. 15. Hyp: Hyperplasia tissue. Ca: Carcinoma tissue. WT=Akt1^{+/+} genotype. KO=Akt1^{-/-} genotype.

FIG. 17 provides exemplary data showing cyclin D1 expression in PyMT Akt^{+/+} and PyMT Akt1^{-/-} transgenic mice. Panel A: Twelve week old Akt^{+/+} (hyperplasia) (upper) versus twelve week old Akt^{-/-} no (hyperplasia) (lower). Panel B: Twenty seven week old Akt1^{-/-} (hyperplasia). Panel C: Twelve week old Akt^{+/+} (carcinoma) versus twenty-seven week old Akt^{-/-} (carcinoma).

FIG. 18 provides exemplary data showing PyMT-expression of cyclin D1. Panel A: Six week old Akt2^{+/+} (hyperplasia). Panel B: Six week old Akt2^{-/-} (hyperplasia).

FIG. 19 provides exemplary data showing Tunel positive nuclei in MMTV-PyMT transgenic mice having the following genotypes: Panel A: Akt^{+/+} (hyperplasia versus carcinoma tissue). Panel B: Akt^{-/-} (hyperplasia versus carcinoma tissue).

FIG. 20 provides exemplary data showing that mammary epithelial apoptosis is greater in PyMT transgenic mice having the following genotypes. Panel A: Akt^{+/+}. Panel B: Akt1^{-/-}.

FIG. 21 shows one embodiment of a human AKT1 amino acid sequence (Panel A) and nucleotide sequence (Panel B). (Accession No. NM_{—}001014432).

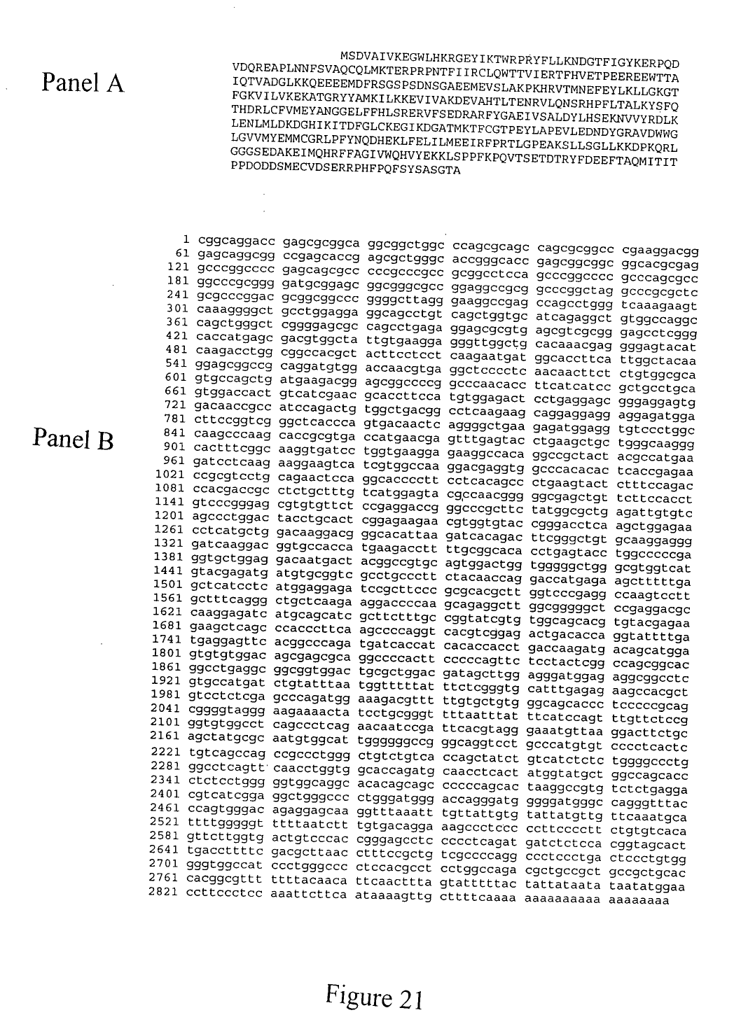

FIG. 22 shows one embodiment of a mouse AKT2 amino acid sequence (Panel A) and nucleotide sequence (Panel B). (Accession No. NM_{—}007434).

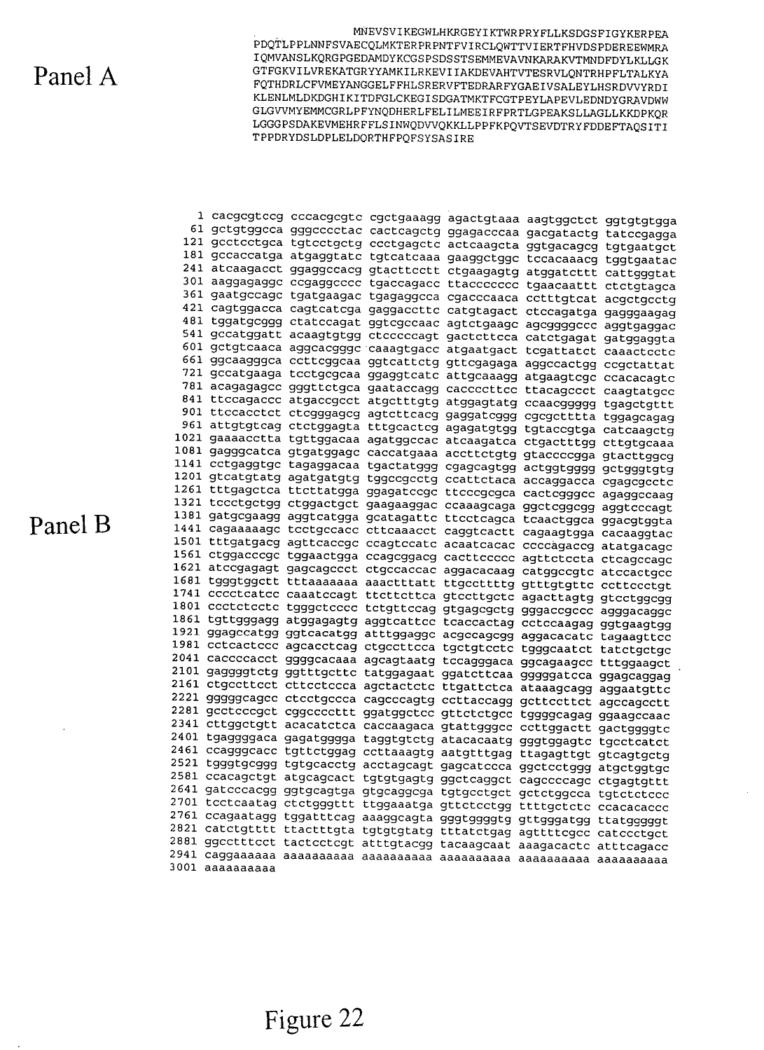

FIG. 23 shows one embodiment of a mouse NEU proto-oncogene promoter region (Accession No. X66236).

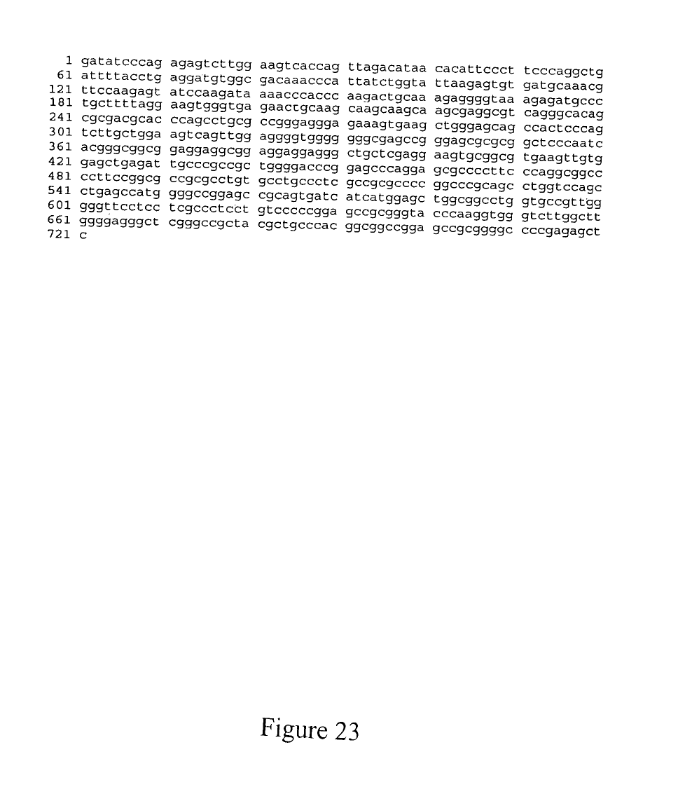

FIG. 24 shows one embodiment of a mouse HER2 oncogene amino acid sequence (Panel A) and nucleotide sequence (Panel B). (Accession No. BC027080).

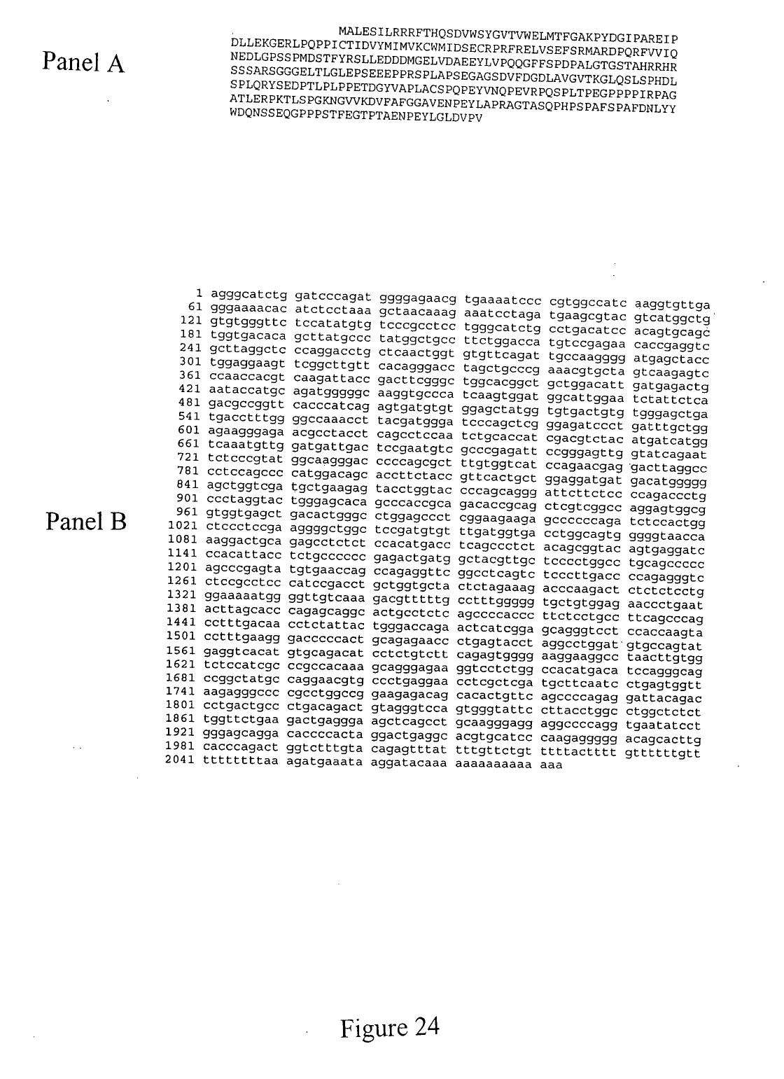

### DETAILED DESCRIPTION OF THE INVENTION

The present invention is related to the development of anti-cancer agents. In one embodiment, the anti-cancer agent regulates the PI3K/Akt pathway. In one embodiment, the anti-cancer agent comprises an Akt protein. In one embodiment, the anti-cancer agent comprises an Akt2 protein. In one embodiment, the anti-cancer agent comprises an Akt1 inhibitor.

I. The PI3K/Akt Pathway and Oncogenesis

A. Akt Overexpression

Akt was first identified as a component of a fusion product of the retroviral oncogene v-akt that causes leukemia in mice but no modified or mutated Akt genes have been found in mammals. Bellacosa et al., Science 254:274-7 (1991). However, a number of studies have discovered PKB/Akt gene overexpression in human cancers. For example, Akt was proposed as a potential human oncogene when detected as overexpressed in a single gastric carcinoma. Staal S. P., Proc Natl Acad Sci USA 84:5034-5037 (1987). PKBb/Akt2 gene overexpression has been found in ovarian, pancreatic, gastric, and breast tumors. Bellacosa et al., Int J Cancer 64:280-285 (1995); Cheng et al., Proc Natl Acad Sci USA 1996; 93:3636-41 (1996). PKBb/Akt2 overexpression was particularly associated with high-grade aggressive ovarian tumors and appears to occur as part of the frequent amplification of the 19q13.1-q13.2 chromosomal region. Thompson et al., Cancer Genet Cytogenet 87:55-62 (1996). PKBg/Akt3 mRNA overexpression and selective activation by growth factors in hormone-independent breast and prostate cancer cell lines has also been reported. Nakatani et al., J Biol Chem 274:21528-21532 (1999). Overall, these studies indicate that PKB/Akt gene overexpression, particularly PKBb/Akt2, may be a frequent occurrence in several human cancers.

B. Activation of Upstream Regulators of the PI-3K/Akt Pathway

The PI-3K/Akt can be activated as a result of the ligand-dependent activation of cell surface receptors including, but not limited to, tyrosine kinase receptors, G-protein-coupled receptors, or integrins. These cell surface receptors are commonly overexpressed, or constitutively active, in many human cancers, thereby activating downstream signaling pathways. Blume-Jensen et al., Nature 411:355-365 (2001).

One of the most extensively studied examples is the erbB2 tyrosine kinase receptor, which is overexpressed in a large number of breast and other cancers. Harari et al., Oncogene 19:6102-6114 (2000). Although erbB2 overexpression is associated with particularly aggressive disease and poor patient prognosis, studies with transgenic mice indicate that erbB2 by itself may be insufficient for transformation. Studies in breast cancer cells, primary breast tumors, and transgenic mice all indicate that erbB2, when overexpressed, is constitutively associated with erbB3. Siegel et al., EMBO J 18:2149-2164 (1999). This provides a basis for data showing that tumor cells overexpressing erbB2 display constitutive PKB/Akt activity. Zhou et al., J Biol Chem 275:8027-8031 (2000). Indeed, recent data indicate that the PKB/Akt pathway may play a role in stimulating proliferation and survival in erbB2-overexpressing cells.

C. IP3 Phosphatases and Akt Regulation

PTEN (also known as MMAC1 or TEP1) is a dual-function lipid and protein phosphatase that was originally identified as a tumor suppressor gene frequently mutated in the advanced stages of a number of human cancers, particularly glioblastoma, endometrial, and prostate cancers. In addition, germline mutations in PTEN give rise to the rare autosomal dominant inherited human cancer syndrome known as Cowden's disease, which is associated with increased risk of developing breast and other cancers. Simpson et al., Exp Cell Res 264:29-41 (2001). The results of studies in which PTEN has been overexpressed in various cell lines suggest that PTEN acts as a tumor suppressor by inhibiting cell growth and/or increasing susceptibility to apoptosis and/or anoikis. Weng et al., Cancer Res 59:5808-5814 (1999); and Lu et al., Oncogene 18:7034-45 (1999). The main physiological lipid substrate for PTEN is phosphoinositoltriphosphate (PIP3), a product of PI-3K. PTEN dephosphorylates PIP3 at the D3 position and thus acts as a negative regulator of PI-3K signaling. Indeed, PTEN-null embryonic fibroblasts display elevated PIP3 levels and constitutive PKB/Akt activity, indicating that PTEN acts to constrain the pathway in unstimulated cells. Stambolic et al., Cell 95:29-39 (1998).

An absence of PTEN also strongly correlates with activation of PKB/Akt in tumor cell lines. Wu et al., Proc Natl Acad Sci USA 95:15587-15591 (1998); and Dahia et al., Hum Mol Genet 8:185-193 (1999). Conversely, reexpression of PTEN in cells lacking PTEN down-regulates PKB/Akt phosphorylation as well as reversing the phosphorylation of PKB/Akt cellular substrates such as BAD. Li et al., Cancer Res 58:5667-5672 (1998); and Davies et al., Cancer Res 58:5285-5290 (1998), respectively. PTEN, through both its lipid and protein phosphatase activities, probably regulates a number of signaling pathways (i.e., for example, the PI3K/Akt signaling pathway), and may contribute to both increased growth and resistance to apoptosis.

Antigrowth effects of PTEN have been suggested to be mediated by both PKB/Akt-dependent and -independent pathways, whereas the proapoptotic effects of PTEN are most likely PKB/Akt-dependent. Weng et al., Hum Mol Genet 10:237-242 (2001). The mechanisms by which PKB/Akt mediates PTEN-induced growth arrest and apoptosis remain unclear but a recent study demonstrated a major role for forkhead transcription factors. Nakamura et al., Mol Cell Biol 20:8969-8982 (2000).

D. HER2/Neu Overexpression

HER2/Neu is a member of the epidermal growth factor receptor family. Overexpression of HER2/Neu is observed in approximately one third of breast cancers and represents a poor prognostic sign. Overexpression of HER2/Neu activates multiple signaling pathways including, but not limited to, the PI3K/Akt pathway. The PI3K/Akt pathway is also activated by the Polyoma Middle T antigen (PyMT). Expression of either HER2/Neu or PyMT in the mammary gland of transgenic mice comprising MMTV LTR-driven transgenes give rise to mammary adenocarcinomas.

In some embodiments, the present invention contemplates a role for Akt-regulation of the oncogenic potential of the PI3K/Akt pathway that contradicts that which is generally believed in the art (most data, of which, was performed using in vitro models). Specifically, the art teaches that an Akt2 knockout mouse would be expected to inhibit the development of mammary epithelial tumors (i.e., Akt2 is generally believed to be a tumor promoter). Also, the art teaches that an Akt1 knockout mouse would be expected to enhance the development of mammary epithelial tumors (i.e., Akt1 is generally believed to be a tumor suppressor). The data presented herein teaches that, in fact, the opposite effects are observed when performing in vivo studies.

For example, one previously reported study has shown that whereas the ablation of Akt2 inhibits the proliferation and survival of MCF7 cells in 3D cultures, the ablation of Akt1 induces EMT and promotes cell migration in response to IGF-I and EGF. Further, the phenotypic effects of Akt1 ablation have been attributed to the activation of the ERK pathway. Another previously reported study showed that Akt1 promotes the degradation of the NFAT via the HDM2 E3 ubiquitin ligase. Ablation of Akt1, therefore, would be predicted by the art to promote the upregulation of NFAT, which enhances the expression of COX2 and stimulates cell migration (i.e., promotes metastases).

The present invention directly studies the problem of tumor development using in vivo models. This approach is unlike that taken in the previously reported studies that used in vitro cell culture models. Alternatively, other factors may explain the differences between embodiments contemplated herein and those reported previously including, but not limited to: a) the nature of the cells undergoing transformation (i.e., for example, myoepithelial versus lobular or ductal mammary epithelia); and b) the cell culture systems previously used are believed oversimplified in that they lack the complex interactions characteristic of in vivo systems in intact animals. Specifically, these cell culture systems: i) lack the full range of the interactions between the stroma and the epithelial cells; ii) are devoid of the immune system; and iii) lack hormonal and other factors that function from a distance within a living organism.

In some embodiments, the present invention contemplates the role of Akt in mammary oncogenesis. In other embodiments, the present invention contemplates the role of Akt in non-mammary oncogenesis. In one embodiment, Akt plays a role in the development of thymomas.

Two lines of transgenic mice have been generated to show that Akt regulates the development of thymic tumors. In one embodiment, a transgenic wild-type mouse constitutively expresses active Akt in the thymus and concomitantly develops thymomas. In one embodiment, a transgenic Akt knockout mouse fails to develop thymomas. Similar, to the data herein discussing mammary gland tumors, the dynamic nature of Akt oncogenic regulation is dependent upon the specific Akt isoform.

Transgenic mice expressing MyrAkt from a proximal Lck promoter construct develop thymomas at an early age, whereas transgenic mice expressing constitutively active Lck-AktE40K develop primarily tumors of the peripheral lymphoid organs later in life. Previous studies support the hypothesis that MyrAkt-induced tumors appear when organ size control mechanisms operating in the thymus fail to down-regulate cyclin D3 expression and block G1 progression promoted by MyrAkt.

Oncogenic mutations associated with the majority of human cancers are believed to activate Akt. Although the growth and metastatic potential of many of these cancers may depend on the continuing activity of Akt, it was not known, until now, whether all Akt isoforms (i.e., for example, Akt1, Akt2 and Akt3) play an equally important role in the transduction of the oncogenic signals induced by these mutations.

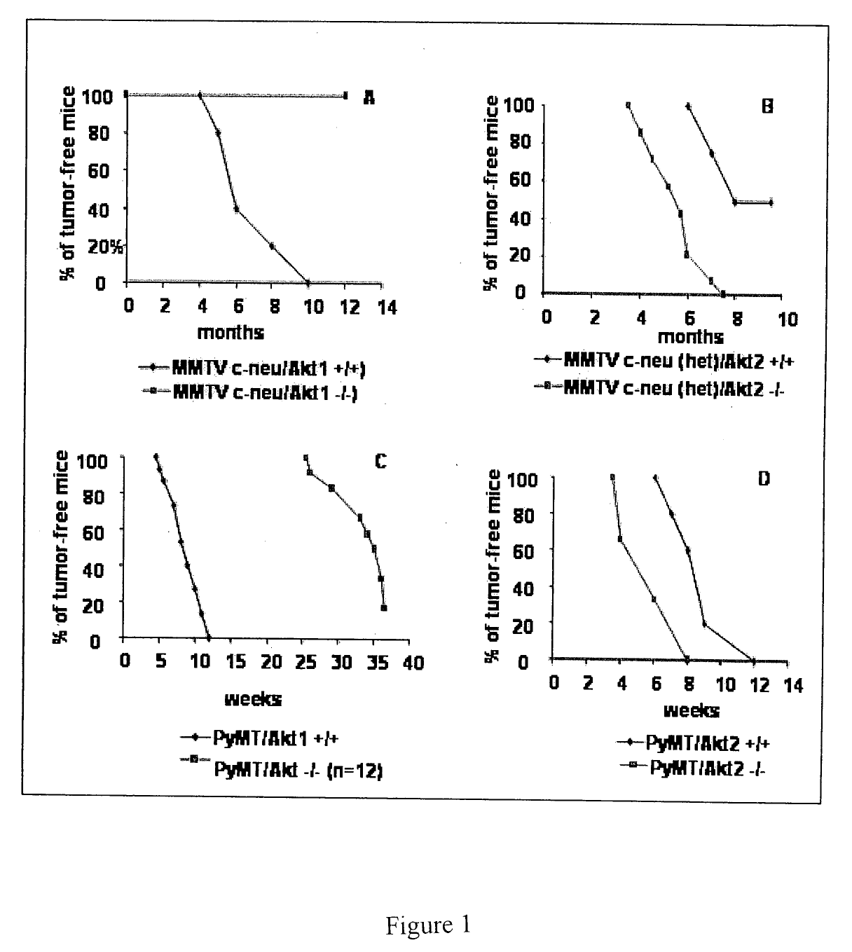

In one embodiment, the present invention contemplates that Akt isoform specificity in the transduction of oncogenic signals provides for a role of Akt in the development and the biology of MMTV-HER2/Neu and MMTV-PyMT-induced mammary adenocarcinomas in mice. Preliminary data showed that ablation of Akt1 (i.e., for example, using an Akt1^{-/-} transgenic mouse) significantly inhibits, while ablation of Akt2 (i.e., for example, using an Akt2^{-/-} transgenic mouse), and perhaps Akt3, accelerates the development of mammary tumors in MMTV-HER2/Neu and MMTV-PyMT transgenic mice. See FIG. 1.

II. Akt Serine-Threonine Protein Kinase

A. Structural Features of PKB/Akt Proteins

The PKB/Akt story began with the isolation of two genes, named Akt1 and Akt2, which were identified as human homologues of the viral oncogene v-akt, previously known to cause a form of leukemia in mice. Staal S. P., Proc Natl Acad Sci USA 84:5034-5037 (1987). Subsequently, three independent studies revealed that v-akt and its mammalian homologues encoded a protein kinase with some similarities to protein kinase C (PKC) and protein kinase A (PKA). Bellacosa et al., Science 254: 274-277 (1991); Coffer et al., Eur J Biochem 201:475-81 (1991); and Jones et al., Proc Natl Acad Sci USA 88:4171-4175 (1991). To date, three members of the family have been isolated and these are now referred to as PKBa (Akt1), PKBb (Akt2), and PKBg (Akt3). They are products of distinct genes but are highly related, exhibiting greater than 80% homology at the amino acid level. The three genes are expressed differentially, with PKBa/Akt1 and PKBb/Akt2 displaying fairly broad and PKBg/Akt3 more restricted tissue distribution.

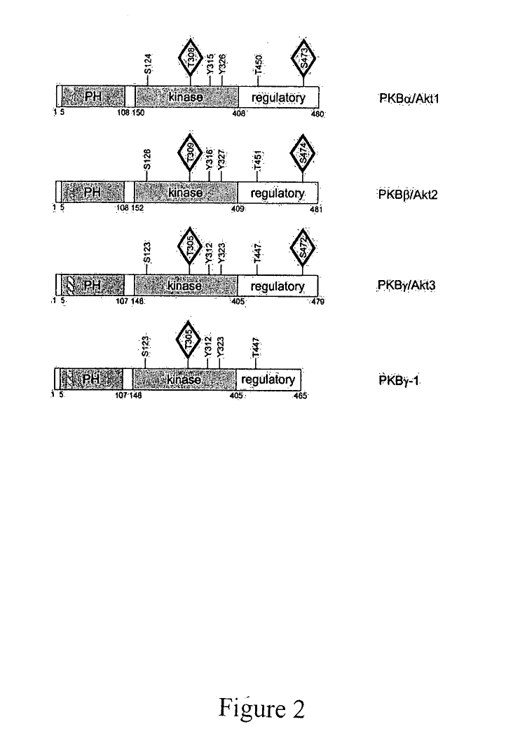

Each PKB/Akt isoform possesses an N-terminal pleckstrin homology (PH) domain of approximately 100 amino acids. See FIG. 2. Each isoform consists of an N-terminal PH domain containing a region (shown in hatched bars) for binding inositol phospholipids, a kinase domain, and a C-terminal regulatory domain. Residues contained within diamonds are serine and threonine sites inducibly phosphorylated in response to various cell stimuli. Other residues in PKBa/Akt1 known to be phosphorylated constitutively but not thought to regulate catalytic activity are also highlighted. Tyrosine residues Y315 and Y326 have recently been described as having a potential role in the regulation of PKBa/Akt1 activity. The equivalent phosphorylation sites in PKBb/Akt2, PKBg/Akt3, and PKBg1 have been obtained from sequence databases. Recent detailed structural examination of PKB/Akt PH domains reveals similarity to PH domains found in other signaling molecules that bind 3-phosphoinositides. Lietzke et al., Mol Cell 6:385-394 (2000); and Ferguson et al. Mol Cell 6:373-384 (2000). This, together with evidence from earlier in vitro studies, indicates that the PH domain mediates binding of PKB/Akt to 3-phosphoinositides. The PH domain is followed by the kinase catalytic domain, which shows a high degree of similarity to those found in PKA and PKC. Jones et al., Cell Regul 2:1001-9 (1991); and Andjelkovic et al., J Biol Chem 1270:4066-4075 (1995). Also present in this region is a threonine residue (T308 in PKBa/Akt1) whose phosphorylation is necessary for activation of PKB/Akt. Following the kinase domain is a hydrophobic C-terminal tail containing a second regulatory phosphorylation site (S473 in PKBa/Akt1). Phosphorylation at T308 and S473 occurs in response to growth factors and other extracellular stimuli and is essential for maximal PKB/Akt activation PKB/Akt may also be phosphorylated on S1124 and T450 but neither of these sites appears to regulate PKB/Akt activity and their phosphorylation does not change following cell stimulation. Alessi et al., EMBO J 15:6541-6551 (1996). Splice variants of PKBg/Akt3 lacking the Ser⁴⁷² phosphorylation site have been identified Konishi et al., Biochem Biophys Res Commun 216:526-34 (1995). The human variant, PKBg1, appears to be regulated differently from that of full-length PKBg/Akt3 in a manner that suggests that the absence of the second phosphorylation site limits its maximal potential for both membrane translocation and catalytic activation. Brodbeck et al., J Biol Chem 276:29550-29558 (2001).

Akt is a serine threonine protein kinase that is activated by a variety of stimuli via PI3K-dependent mechanisms. Akt is believed to regulate a variety of cellular functions including survival, proliferation and intermediary metabolism via the PI3K pathway. There are three Akt isofomms, which are very similar in sequence. In tissue culture experiments, all three isoforms appear to be regulated similarly and to have overlapping targets. However, in animal studies, the ablation of individual Akt isoforms gives rise to distinct phenotypes.

In one embodiment, the present invention contemplates a transgenic mouse comprising an Akt mutated transgene. In one embodiment, the Akt mutated transgene generates an Akt1^{-/-} mouse which is small and exhibits perinatal lethality. In one embodiment, the Akt mutated transgene generates an Akt2^{-/-} mouse which develops insulin-resistant diabetes. In one embodiment, the Akt mutated transgene generates an Akt3^{-/-} mouse which has small brains and exhibits mild neurological defects. In other embodiments, Akt2^{-/-} and/or Akt3^{-/-} mice are viable and fertile, whereas Akt1^{-/-} and/or Akt2^{-/-} mice die perinatally with severe musculoskeletal and adipose tissue defects and Akt1^{-/-}/Akt3^{-/-} mice die prematurely (i.e., for example, at E9.5−E10.5; E=embryonic development day) with severe neurological and cardiac defects.

Although it is not necessary to understand the mechanism of an invention, it is believed that phenotypic differences seen upon ablation of individual Akt isoforms (i.e., for example, by the generation of a knock-out mouse) may have a major translational impact. It is further believed that, these knockout mouse phenotypes may demonstrate how Akt inhibitors can be used in the treatment of human cancer.

Recent studies report that ablation of Akt1 activates ERK and promotes the overexpression of NFAT and COX2 in cultured human mammary epithelial cells. The resultant changes in the signaling pathway in turn, are suggested to promote epithelial-mesenchymal transition (EMT) and enhances cell migration. The art believes that these data suggest that selective inhibition of Akt1 may have undesirable effects in the course of human breast cancer. This belief is contrary to specific embodiments contemplated by the present invention.

These preceding studies demonstrating the signaling specificities of individual Akt isoforms however, did not address any translational significance. Thus, these studies do not predict whether these cells are transformed in the course of human mammary oncogenesis. More important, these studies were carried out on cultured cells, removed from the complex environment of the intact animal. The present invention solves these problems by demonstrating the specificity of individual Akt isoforms in intact animals.

In one embodiment, the present invention contemplates a method comprising crossing MMTV-HER2/Neu and MMTV-PyMT transgenes into Akt1^{-/-}, Akt2^{-/-}, and/or Akt3^{-/-} genetic backgrounds. In one embodiment, these crossings generate knockout mice. In one embodiment, wild type littermates of the knockout mice, harboring the oncogenic control transgenes, are monitored for the development of mammary adenocarcinomas. In one embodiment, a mouse comprising an Akt1 knockout allele has inhibited tumor development. Although it is not necessary to understand the mechanism of an invention, it is believed that these data suggest that the Akt1 isoform protein functions as a tumor promoter. In one embodiment, a mouse comprising an Akt2 knockout allele has accelerated tumor development. Although it is not necessary to understand the mechanism of an invention, it is believed that these data suggest that the Akt2 isoform protein functions as a tumor suppressor.

Histologically, tumors developing in the double allelic Akt1^{-/-} knockout mouse line, and to a lesser extent, in the double allelic Akt3^{-/-} knockout mouse line comprise smaller neoplastic cell islands interspersed with adipose tissue and the stroma contains more fibrous tissue than the control phenotype. On the other hand, tumors developing in Akt2^{-/-} mice were cystic and exhibited a papillary histologic phenotype. All Akt genotypes developed tumors that were invasive, but the frequency of distant metastases was significantly lower in the Akt1^{-/-} mice. Although it is not necessary to understand the mechanism of an invention, it is believed that the ablation of individual Akt isoforms did not affect mammary gland development during puberty, or the expression of the transgenes (i.e., for example, regulation of the promoter). It is further believed that the observed phenotypes were due to differential roles of individual Akt isoforms in the transduction of the oncogenic signals induced by the transgenes. Additional studies indeed revealed that both cell proliferation and cell survival were impaired in the early stages of tumorigenesis in Akt1 knockout mice. Moreover, tumors developing in Akt1 knockout mice exhibited large focal areas of apoptosis.

III. Oncogenes Affecting the PI3K/Akt Pathway

Activation of the PI3K/Akt pathway is believed to be very common in human breast cancer. Although it is not necessary to understand the mechanism of an invention, it is believed that PI3K/Akt activation is most likely the result of mutations in genes whose products regulate, directly or indirectly, the synthesis and/or degradation of D3 phosphorylated phosphoinositides (D3 PPIs). One gene whose mutation might activate the PI3K/Akt pathway is the HER2/Neu oncogene, which encodes a member of the epithelial growth factor (EGF)-receptor (EGF-R) family of tyrosine kinases.

Over-expression of HER2/Neu occurs in approximately one third of human breast cancer cases and is associated with poor prognosis. The oncogenic potential of HER2/Neu has been confirmed using animal models. Thus, HER2/Neu over-expression from a MMTV LTR-driven transgene in mice, gives rise to mammary adenocarcinomas which originate in glandular epithelium and are histologically similar to a significant fraction of human breast cancers. Another MMTV promoter driven transgene, MMTV-PyMT, also activates the PI-3K/Akt pathway and gives rise to mammary adenocarcinomas in mice. The tumors induced by PyMT are histologically similar to the ones induced by the HER2/Neu transgene, however, PyMT tumors have a shorter latency and are more aggressive and metastatic.

Overexpression of both the HER2/Neu and MMTV-PyMT oncogenes in the murine mammary gland from MMTV-LTR driven transgenes is sufficient to induce mammary adenocarcinomas having similarity to the human disease. Data presented herein shows that the HER2/Neu and MMTV-PyMT oncogenic transgenes can be modified to generate embodiments of the present invention comprising: i) a transgenic mouse line comprising a knockout of at least one Akt1 allele demonstrating inhibited tumor development; and ii) a transgenic mouse line comprising a knockout of at least one Akt2 allele demonstrating accelerated tumor development. In other embodiments, tumors arising in wild type transgenic mice comprising an HER2/Neu and/or MMTV-PyMT oncogenic transgene express high levels of Akt1 and low levels of Akt2. Despite the histological differences between the Akt1^{-/-}, Akt2^{-/-}, Akt3^{-/-} and wild type mice tumors, all are were demonstrated as invasive. The frequency of metastasis, however, was observed to be significantly lower in tumors arising in Akt1 knockout mice. The development of the mammary gland during puberty in all Akt1 knockout mice versus wild type mice was seen to be indistinguishable. Moreover, the expression of the transgene is not affected by the ablation of various Akt isoforms (i.e., Akt knockout does not effect promoter regulation). Although it is not necessary to understand the mechanism of an invention, it is believed that these combined data suggest that the observed differences in HER2/Neu or PyMT-induced tumorigenesis in the Akt1^{-/-}, Akt2^{-/-} and Akt3^{-/-} genetic backgrounds is due to differences in the signaling specificities of the three Akt isoforms.

IV. Akt Regulation of Apoptosis

The intrinsic capacity of all cells to undergo apoptosis is suppressed by survival signals induced by factors within their immediate microenvironment. Studies have repeatedly shown that PKB/Akt is involved in cell survival triggered by growth factors, extracellular matrix, and other stimuli. Datta et al., Genes Dev 13:2905-2927 (1999). For example, dominant negative alleles of PKB/Akt reduce the ability of growth factors and other stimuli to maintain cell survival whereas overexpression of wild type or activated Akt can rescue cells from apoptosis induced by various stress signals. Kennedy et al., Genes Dev 11:701-713 (1997); Khwaja et al., EMBO J 16:2783-2793 (1997); and Kulik et al., Mol Cell Biol 17:1595-1606 (1997).

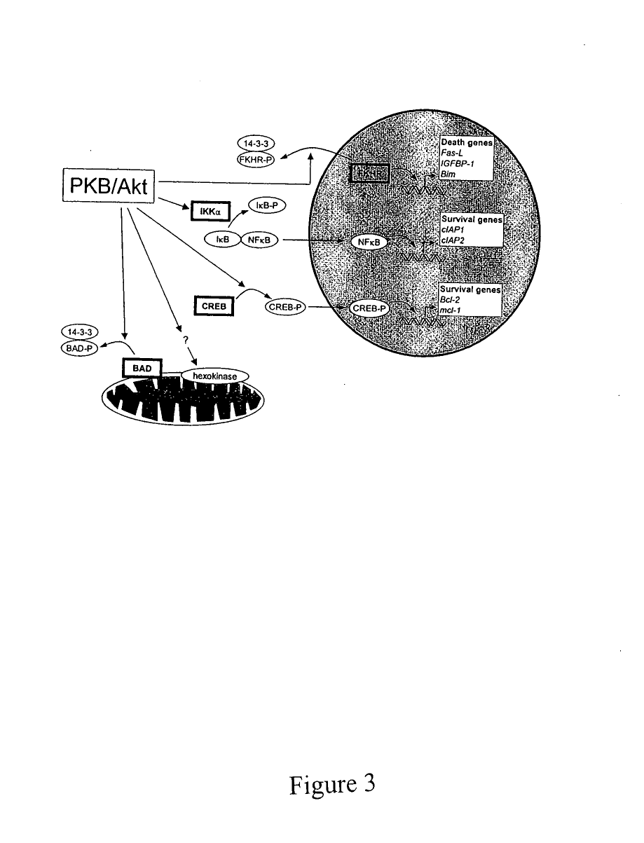

Targets of PKB/Akt signaling that are believed to promote cell survival are illustrated herein. See FIG. 3. PKB/Akt promotes cell survival by multiple mechanisms: (1) decreasing the transcription of death genes by phosphorylating forkhead family transcription factors such as FKHR, which promotes their sequestration by 14-3-3 proteins in the cytoplasm, (2) increasing the transcription of survival genes by activating NF-kB and CREB transcription factors, (3) phosphorylating and inactivating the proapoptotic protein BAD, and (4) maintaining mitochondrial integrity by activating hexokinase. Although substrates are placed in particular subcellular localizations, in most cases, the location in which PKB/Akt phosphorylation takes place is uncertain. Numerous studies indicate that PKB/Akt promotes survival by directly phosphorylating key regulators of the apoptotic cascade. The most widely studied example of this type of regulation involves BAD, a member of the Bcl-2 family, which promotes apoptosis by binding to and antagonizing the actions of prosurvival members of the family such as Bcl-2 and Bcl-XL. PKB/Akt can phosphorylate BAD at residue S136 and this modification promotes the sequestration of BAD by 14-3-3 proteins in the cytosol, thus preventing BAD from interacting with Bcl-2 or Bcl-XL at the mitochondrial membrane. del Peso L et al., Science 278:687-69 (1997); and Datta et al., Cell 91:231-241 (1997). PKB/Akt-induced phosphorylation of BAD may also occur indirectly, through intervening protein kinases such as Raf-1 and p65PAK. These and other studies indicate that PKB/Akt-dependent phosphorylation of BAD is linked to the promotion of cell survival in some cellular contexts.

Most potential PKB/Akt targets are believed to regulate apoptosis prior to the release of cytochrome c from the mitochondria and activation of the caspase cascade that characterizes the terminal execution phase of apoptosis. However, data suggesting that PKB/Akt may also influence postmitochondrial events have also been reported. Procaspase-9, the initiator caspase in the caspase cascade, was shown to be a substrate for PKB/Akt. Cardone et al., Science 282:1318-1321 (1998). In these experiments, phosphorylation of human procaspase-9 by PKB/Akt blocked its intrinsic protease activity and was shown to provide an overall regulation of the apoptotic process. Exogenous PKB/Akt has also been suggested to inhibit the activation of caspases-9 and -3 induced by cytochrome c in a cell-free system. Zhou et al., J Cell Biol 151:483-494 (2000).

A. Small Molecule Inhibitors

AKT kinases have been selected as targets for anticancer therapeutics at many academic and pharmaceutical institutions. AKT kinases have undergone considerable small molecule drug discovery because of their potential role in tumor cell survival/proliferation and their overexpression/activation in many human cancers. Many studies support a general rationale for targeting AKT kinases in new drug discovery efforts. Consequently, the structural features of AKT kinase in its inactive and active states, as determined by crystal structure analysis, were analyzed and reported. Such small molecule inhibitors may target the ATP binding site, PH domain and protein substrate binding site, as well as isoform selective allosteric inhibitors. Kumar et al., “AKT crystal structure and AKT-specific inhibitors” Oncogene 14:7493-501 (2005).

Recent advances in the development and biological evaluation of small molecule inhibitors for the serine/threonine kinase Akt (PKB) has shown that Akt plays a role in cell survival and proliferation through a number of downstream effectors. Unregulated activation of the PI3K/Akt pathway is observed in many human cancers and Akt is over-expressed or activated in all major cancers. Consequently, Akt is considered an attractive target for chemotherapy and it has been postulated that inhibition of Akt alone, or in combination with standard cancer chemotherapeutics, will reduce the apoptotic threshold and preferentially kill cancer cells. Small molecule Akt inhibitors are generally ATP-competitive inhibitors providing little specificity. For example, phosphatidylinositol (PI) analogs have been reported to inhibit Akt, but these inhibitors may also have specificity problems with respect to other pleckstrin homology (PH) domain-containing proteins and may result in poor bioavailability. None of the inhibitors in these classes have been reported to have Akt isozyme specificity. Barnett et al., “The Akt/PKB family of protein kinases: a review of small molecule inhibitors and progress towards target validation” Curr Top Med Chem. 5(2):109-25 (2005).

Small molecule inhibitors of Akt having pleckstrin homology domain-dependent, isozyme-specific activity showing isozyme specific inhibition that effect the apoptotic response of tumor cells to a variety of chemotherapies has been reported. Using multiple cell backgrounds, maximal induction of caspase-3 activity was achieved when both Akt1 and Akt2 were inhibited. This induction was not reversed by overexpression of functionally active Akt3. The level of caspase-3 activation achieved under these conditions was equivalent to that observed with the phosphatidylinositol-3-kinase inhibitor LY294002. DeFeo-Jones et al., “Tumor cell sensitization to apoptotic stimuli by selective inhibition of specific Akt/PKB family members” Mol Cancer Ther. 4:271-279 (2005).

Akt is reported to transduce survival signals from survival/growth factors. Consequently, a deregulation and signal imbalance in the cancer cell Akt pathway facilitates apoptosis. Upregulation or activation of Akt is implicated to support the survival of cancer cells. Small-molecule Akt inhibitors that inhibit Akt activity and block phosphorylation by Akt on multiple downstream targets in cells have been reported. A synergistic response was seen when Akt inhibitors were combined with other known apopototic agents such as paclitaxel, doxorubicin, camptothecin or enhancement of topoisomerase inhibitor cytotoxicity. Shi et al., “Optimal Classes of Chemotherapeutic Agents Sensitized by Specific Small-Molecule Inhibitors of Akt In Vitro and In Vivo” Neoplasia 7:992-1000 (2005).

A high-throughput HTRF (homogeneous time-resolved fluorescence) assay for Akt kinase activity was reported which screened approximately 270,000 compounds for their ability to inhibit the three isoforms of Akt. Of these, only two Akt reversible inhibitors were identified that exhibited isoenzyme specificity. The first compound (Akt-I-1) inhibited only Akt1 (IC₅₀=4.6 μM), while the second compound (Akt-I-1,2) inhibited both Akt1 and Akt2 with IC₅₀ values of 2.7 and 21 μM, respectively. In addition to inhibiting kinase activity of these individual Akt isoforms, both inhibitors blocked the phosphorylation and activation of the corresponding Akt isoforms by PDK1 (phosphoinositide-dependent kinase 1). Further, these Akt inhibitors were seen to promote TRAIL (tumor-necrosis-factor-related apoptosis-inducing ligand)-induced apoptosis in LNCap prostate cancer cells. Barnett et al., “Identification and characterization of pleckstrin-homology-domain-dependent and isoenzyme-specific Akt inhibitors” Biochem J 385:399-408 (2005).

The identification of several series of Akt small molecule inhibitors have been reported using an iterative analog library synthesis technique. These inhibitors were effective at inducing apoptosis in tumor cells and inhibiting Akt phosphorylation in vivo. Lindsley et al., “Allosteric Akt (PKB) inhibitors: discovery and SAR of isozyme selective inhibitors” Bioorg Med Chem Lett. 1; 15(3):761-4 (2005); and Zhao et al., “Discovery of 2,3,5-trisubstituted pyridine derivatives as potent Akt1 and Akt2 dual inhibitors” Bioorg Med Chem Lett. 15; 905-9 (2005).

B. Antibodies

Anti-Akt1 single-chain antibodies (scFv) have been developed by panning a mouse phage-displayed scFv recombinant antibody library. Recombinant scFv that bound glutathione S-transferase (GST)-Akt1 were screened for their ability to inhibit Akt activity in vitro in a kinase reaction containing human recombinant Akt1 and an Akt/serum glucocorticoid-inducible kinase (SGK) substrate. Michaelis-Menten analysis of kinase inhibition by a selected scFv was consistent with scFv-mediated competition with enzyme's substrate for the catalytic site of Akt. Phosphorylated (activated) phosphoinositide-dependent kinase 1, mitogen-activated protein kinase, p38, and HER2 (erbB2) were not affected, supporting Akt kinase specificity for the inhibitory scFv. Exogenously expressed, constitutively active, Akt2 and Akt3 were also inhibited in vitro by an anti-Akt1 fusion protein. Furthermore, GST-anti-Akt1-MTS fusion protein induced apoptosis in three cancer cell lines that express constitutively active Akt. Finally, systemic treatment with the anti-Akt scFv reduced tumor volume and neovascularization and increased apoptosis in PyVmT-expressing transgenic tumors implanted in mouse dorsal window chambers. Thus, GST-anti-Akt1-MTS is a novel cell-permeable inhibitor of Akt, which selectively inhibits Akt-mediated survival in intact cells both in vitro and in vivo. Shin et al. “Proapoptotic activity of cell-permeable anti-Akt single-chain antibodies” Cancer Res. 65:2815-24 (2005).

V. Transgenic Mice Lines

PKB/Akt is believed to be involved in many signaling pathways that could have a major impact on processes that are abnormally regulated in cancer. For example, the frequency of activation or overexpression of PKB/Akt is prevalent in human cancers. The question of whether PKB/Akt itself is tumorigenic has been addressed by overexpressing the protein in various host systems.

Forced overexpression of wild type PKBb/Akt2 was shown in one study to transform NIH-3T3 fibroblasts. Cheng et al., Oncogene 14:2793-2801 (1997). Transformation, however, did not occur in Rat-1 fibroblasts when exposed to PKBb/Akt2 overexpression. Mirza et al., Cell Growth Differ 11:279-292 (2000). Further, rodent wild type PKB/Akt isoforms also failed to cause transformation of chicken embryo fibroblasts. Nevertheless, membrane targeted, constitutively active versions of each PKB/Akt isoform are sufficient for oncogenic transformation of chicken embryo fibroblasts. Aoki et al., Proc Natl Acad Sci USA 95:14950-14955 (1998); and Mende et al., Oncogene 20:4419-4423 (2001). The observations indicate that in vitro studies are inconsistent, thereby suggesting that tumorigenic studies are more advantageously studies in in vivo models.

A. Akt Transgenic Mice

Although it is not necessary to understand the mechanism of an invention, it is believed that the pathophysiological mechanism by which Akt1 ablation inhibits tumorigenesis, indicates that the loss of Akt1-transduced signals inhibits cell proliferation and promotes apoptosis in pre-neoplastic and early neoplastic regions. Moreover, it is further believed that tumors arising in Akt1 knockout mice contain focal areas of enhanced apoptosis.

One hypothesis suggests that these apoptotic areas develop because of hypoxia in sections of the developing tumor. Alternatively, the more frequent appearance of apoptotic areas in Akt1 knockout mice tumors could be either the result of impaired angiogenesis in the Akt1^{-/-} tumors, or the result of a more pronounced apoptotic response in hypoxic areas of the tumor in Akt1^{-/-} mice. In one embodiment, the present invention contemplates that an Akt1 knockout mouse line comprises reduced cell proliferation and increased apoptosis, thereby resulting in a slower growth rate for preneoplastic and neoplastic foci when compared to wild type mice harboring an oncogenic transgene (i.e., for example, an HER2/Neu and/or MMTV-PyMT oncogenic transgene).

Consequently, data collected using transgenic mice has shown consistency in early studies of the role of PKB/Akt in the transformation of epithelial cells. Targeted overexpression of PKBa/Akt1 in the mammary gland caused hyperplasia but not dysplasia or neoplasia. Ackler et al., Proc Amer Assoc Cancer Res 41:405 (2000). Transgenic mice expressing a mammary gland-targeted, constitutively active PKB/Akt, wherein both regulatory phosphorylation sites were mutated to aspartic acid residues, exhibited defects in the normal process of gland involution, but did not develop tumors. Schwertfeger et al., Mol Endocrinol; 15:867-81 (2001); and Hutchinson et al., Mol Cell Biol 21:2203-2212 (2001) respectively. However, crossing of these animals with mice bearing a mammary gland-targeted mutant middle T antigen uncoupled from PI-3K, produced mice that developed tumors more rapidly than those animals with mutant middle T alone. Interestingly, strong phosphorylation of FKHR and induction of cyclin D was observed in tumors from the crossed animals but not in the mice bearing activated PKB/Akt alone. The cross-bred mice also did not develop metastatic tumors, suggesting that additional events are required for metastatic progression. Overall, the phenotypes of PKB/Akt transgenic mice suggested that mere overexpression of PKB/Akt is not tumorigenic. Constitutive activation of PKB/Akt may be sufficient for tumor formation in some contexts but it is more likely that activated PKB/Akt cooperates with other oncogenic pathways to promote tumor progression.

B. p130Cas Transgenic Mice

MMTVp130Cas transgenic mice were developed to study extensive mammary epithelial hyperplasia seen during pregnancy and delayed involution at the end of lactation. To investigate the mechanisms through which p130Cas adaptor protein is linked to tumorigenesis, we generated mouse mammary tumor virus (MMTV)-p130Cas mice overexpressing p130Cas in the mammary gland. These phenotypes are associated with activation of Src kinase, extracellular signal-regulated kinase 1/2, mitogen-activated protein kinase, and Akt pathways, leading to an increased rate of proliferation and a decreased apoptosis. A double-transgenic line derived from crossing MMTV-p130Cas with MMTV-HER2-Neu mice expressing the activated form of the HER2-Neu oncogene develops multifocal mammary tumors with a significantly shorter latency than the HER2-Neu parental strain alone. Mammary epithelial cells isolated from tumors of double-transgenic mice display increased tyrosine phosphorylation, c-Src, and Akt activation compared with cells derived from HER2-Neu tumors. In addition, p130Cas down-regulation by RNA interference increases apoptosis in HER2-Neu-expressing cells, indicating that p130Cas regulates cell survival. Consistently with the double-transgenic mice model, p130Cas is overexpressed in a significant subset of human breast cancers and high levels of p130Cas in association with HER2 expression correlate with elevated proliferation. These findings provide evidences for a role of p130Cas as a positive regulator of both proliferation and survival in normal and transformed mammary epithelial cells. Its overexpression contributes to HER2-Neu-induced breast tumorigenesis, thus identifying this protein as a putative target for clinical therapy. Cabodi et al., “p130Cas as a new regulator of mammary epithelial cell proliferation, survival, and HER2-neu oncogene-dependent breast tumorigenesis” Cancer Res. 66(9):4672-4680 (2006).

VI. Pharmaceutical Administration

The present invention contemplates pharmaceutical formulations including racemic or optically pure compounds that may be comprised in, but not limited to, powders, capsules, oral or intrapulmonary liquids, tablets, coated tablets, caplets, troches, dispersions, sustained release formulations suspensions, solution, patches and liquids. Young, U.S. Pat. No. 6,369,113 (hereby incorporated by reference). Alternatively, the formulations contemplated in the present invention may be administered intra-nasally. Houdi et al., U.S. Pat. No. 6,150,420 (hereby incorporated by reference).

The above formulations may benefit from increasing the solubility of the drug during delivery to improve absorption. Hydrophilic drugs are usually easily soluble in the natural aqueous environment of a mammal. Hydrophobic drugs, however, are often difficult to dissolve in a manner that provides a steady and predictable delivery to the target organ. Common solubilizers for hydrophobic drugs include, but are not limited to, compounds that contain alcohols, glycols, or esters. Usually, the problem of solving the solubility of hydrophobic drugs involves mixtures containing triglyceride suspensions or colloids. These preparations are acceptable for topical administration but have obvious practical deficiencies when considering the oral or intrapulmonary or intravenous routes. In one embodiment, the present invention contemplates a formulation comprising hydrophobic and hydrophilic surfactants that coat a standard drug delivery device. In one non-limiting example, a bupropion formulation having the hydrophobic/hydrophilic coating is known to dissolve prior to the dispersal of the drug and provides an immediate environment that is highly favorable to solubilizing the drug to facilitate its absorption. Patel et al., U.S. Pat. No. 6,294,192 (hereby incorporated by reference).

The present invention contemplates embodiments having controlled delivery formulations. One example of a controlled delivery formulations is a semi-permeable homopolymer and copolymer film that is water-insoluble, yet water-permeable, and retains an active ingredient within an internal matrix. Preferably, the formulation contains a “water-permeability-modifying agent” within the polymers that changes the rate of osmosis through the polymer. This characteristic thereby controls the exit of the releasable active ingredient retained within the polymer film with the aid of an osmotic enhancing agent. Specifically, an osmotic enhancing agent is a water-soluble material having a high molar water solubility which is capable of achieving, in solution, an osmotic pressure greater than that of the surrounding aqueous environment. These films may be incorporated into standard pharmaceutical preparations such as, but not limited to, tablets, subdermal implants, suppositories, and capsules. Baker et al., U.S. Pat. No. RE33,994 (hereby incorporated by reference).

### EXPERIMENTAL

### Example I

### AKT Isoform Expression in the Mammary Gland

This example demonstrates the relative expression patterns of individual Akt isoforms in the mammary gland.

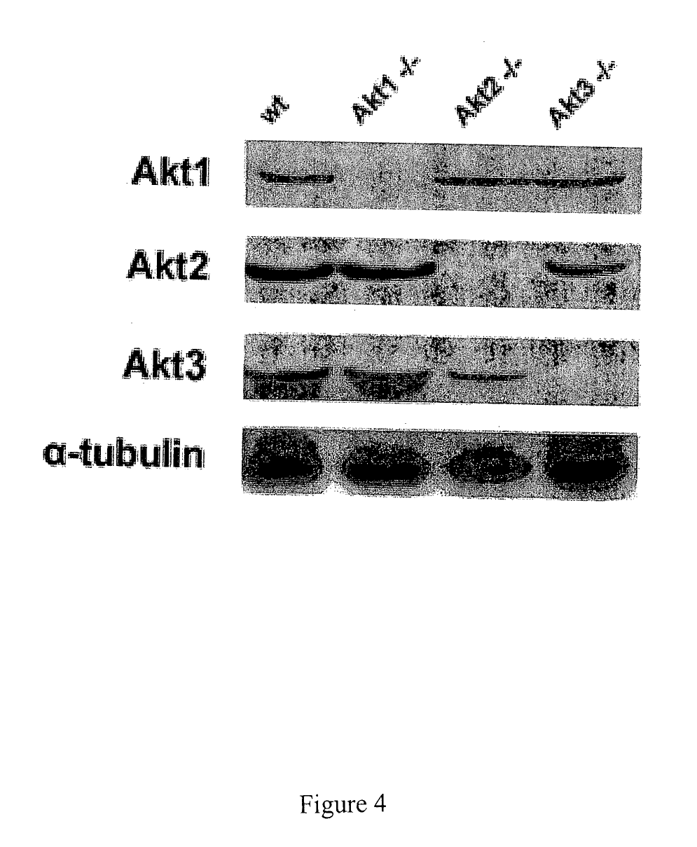

Total cell lysates of mammary tissue from young (6-8 weeks old) wild type, Akt1^{-/-}, Akt2^{-/-} and Akt3^{-/-} mice were Western blotted and probed with antibodies specific for Akt1, Akt2 and Akt3. The results show that all Akt isoforms are expressed at easily detectable levels in the mammary gland. See FIG. 4.

### Example II

### Differential Regulation of Oncogenesis by Akt Isoforms

This example demonstrates that of the three different Akt isoforms, each has a different effect upon mammary oncogenesis.

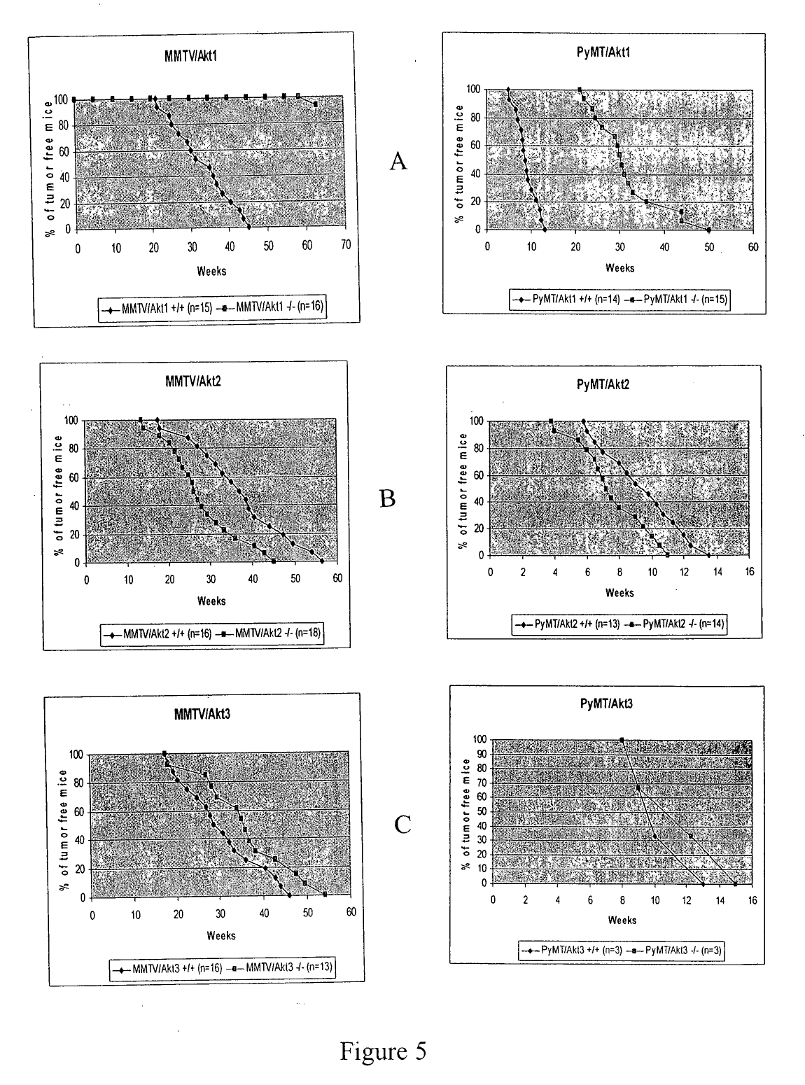

The MMTV-HER2/Neu and the MMTV-PyMT transgenes were crossed into the Akt1^{-/-}, Akt2^{-/-} and Akt3^{-/-} genetic backgrounds. Virgin transgenic mice carrying a single copy of the transgenes were observed for up to 65 weeks. Mice developing mammary adenocarcinomas were sacrificed when the tumors reached 2 cm in diameter and Mayer-Kaplan survival curves were constructed for each mouse strain. See FIG. 5. Since this experiment was carried out in a mixed genetic background, the control mice for each transgenic/Akt knockout strain were Akt^{+/+} littermates of the experimental mice.

The results of this experiment showed that whereas Akt1 ablation inhibits tumor induction, Akt2 ablation accelerates tumor induction. Akt3 ablation has somewhat intermediate effects and appears to only slightly inhibit tumor induction. See FIG. 5.

### Example III

### Akt1 Ablation Inhibits Metastasis

This example demonstrates that the Akt1 isoform may be responsible for mammary gland tumor development and metastasis.

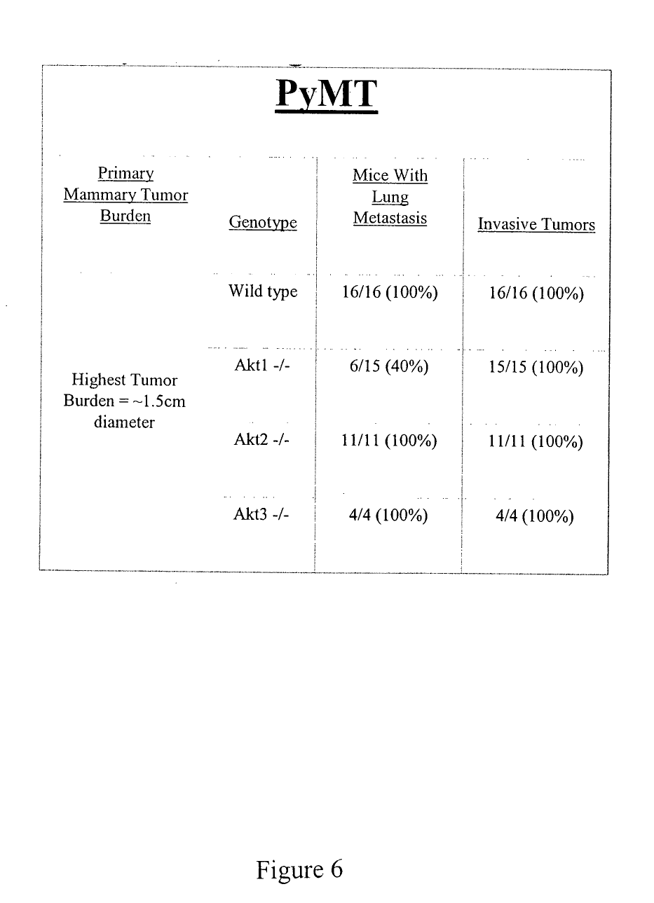

The MMTV-PyMT transgenic mice was used as model because the tumors arising are highly metastatic, with 100% of them developing lung metastases. Serial sections (5 sets, in triplicate) of the lungs of tumor-bearing mice, sacrificed when their tumors reached 2 cm in diameter, were examined both macroscopically and microscopically for metastatic foci. The results revealed that whereas lung metastatic foci could be detected in 100% of wild type, Akt2^{-/-} and Akt3^{-/-} tumor-bearing mice, only 4/14 (28.5%) Akt1^{-/-} mice harbored detectable lung metastases. Local invasiveness of mammary adenocarcinomas, however, was not affected by the ablation of any of the three Akt isoforms, including Akt1. See FIG. 6. Although it is not necessary to understand the mechanism of an invention, it is believed that Akt1 is upregulated, while Akt2 is downregulated in MMTV-HER2/Neu and MMTV-PyMT transgenic mice. One hypothesis suggests that an ablation of Akt1 may inhibit tumor development in MMTV-HER2/Neu and MMTV-PyMT transgenic mice, indicating that an active Akt1 expressed in mammary adenocarcinomas would support tumor development. A second hypothesis suggests that an ablation of Akt2 may promote tumor development in MMTV-HER2/Neu and MMTV-PyMT transgenic mice, indicating that an active Akt2 expressed in mammary adenocarcinomas would suppress tumor development.

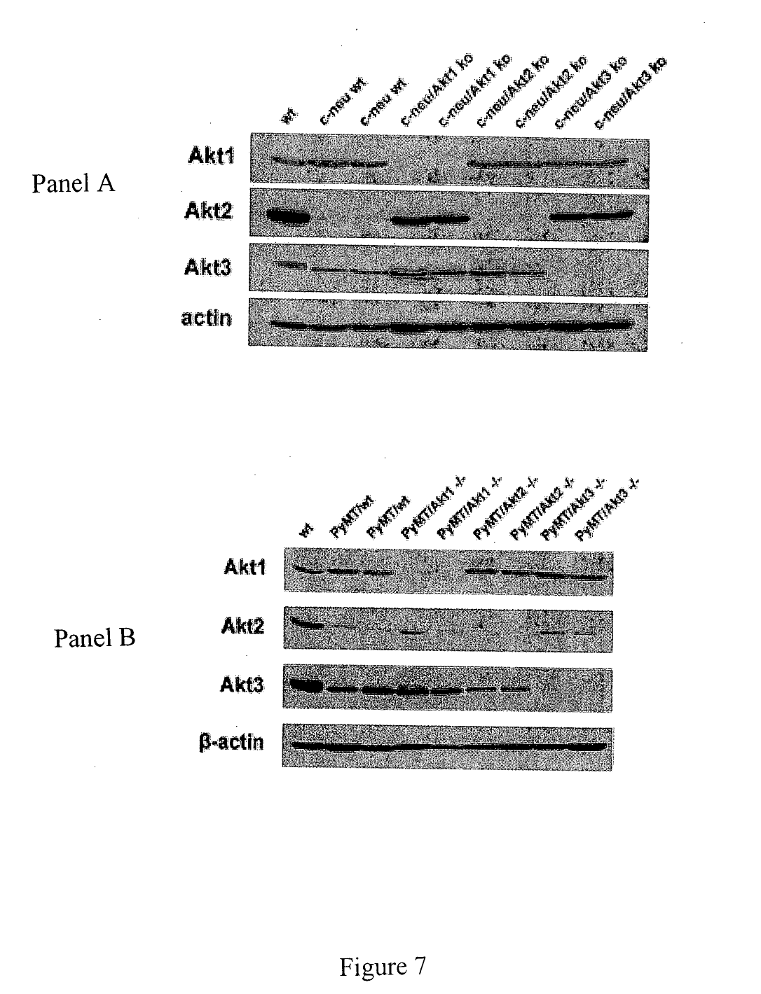

To address these hypotheses, we probed Western blots of tumors arising in MMTV-HER2Neu and MMTV-PyMT transgenic Akt^{+/+} mice with antibodies specific for the three Akt isoforms. Normal mammary gland from 10 week old virgin mice (N=3) was used as a control. To confirm the specificity of the antibodies, we also probed tumor cell lysates from tumors arising in Akt1, Akt2 and Akt3 knockout mice. The results of this experiment showed that whereas Akt1 is upregulated, Akt2 is downregulated in these tumors. Finally, Akt3 is expressed at levels only slightly higher than the ones observed in the normal mammary gland. See FIG. 7. The results of this experiment support the conclusion that the Akt1 isoform protein functions as a tumor promoter and that the Akt2 isoform protein functions as a tumor suppressor.

### Example IV

### Mammary Adenocarcinoma Pathology in Akt Knockout Mice

This example demonstrates that that the differential regulation capability of the several Akt isoforms is reflected in their histological pathology.

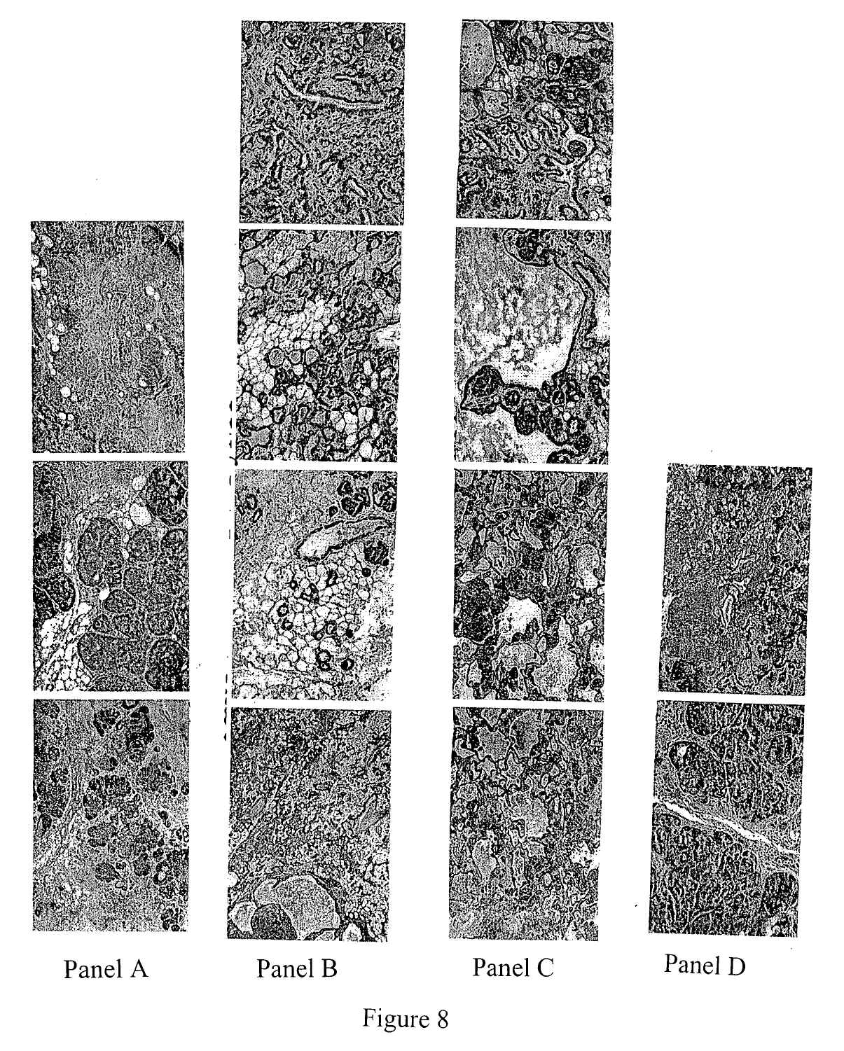

The results of this experiment show that the mammary adenocarcinomas developing in wild type and Akt1, Akt2 and Akt3 knockout mice are histologically distinct. Tumors arising in wild type and Akt3 knockout mice exhibit a characteristic lobular histology. See FIG. 8A and FIG. 8D. Tumors arising in Akt1 knockout mice consist of smaller islands of neoplastic cells, which are interspersed within adipose tissue. See FIG. 8B. The stroma of these neoplastic foci is expanded and consists primarily of fibrous tissue. Finally, the tumors arising in Akt2 knockout mice exhibit a cystic, papillary ductal architecture. See FIG. 8C.

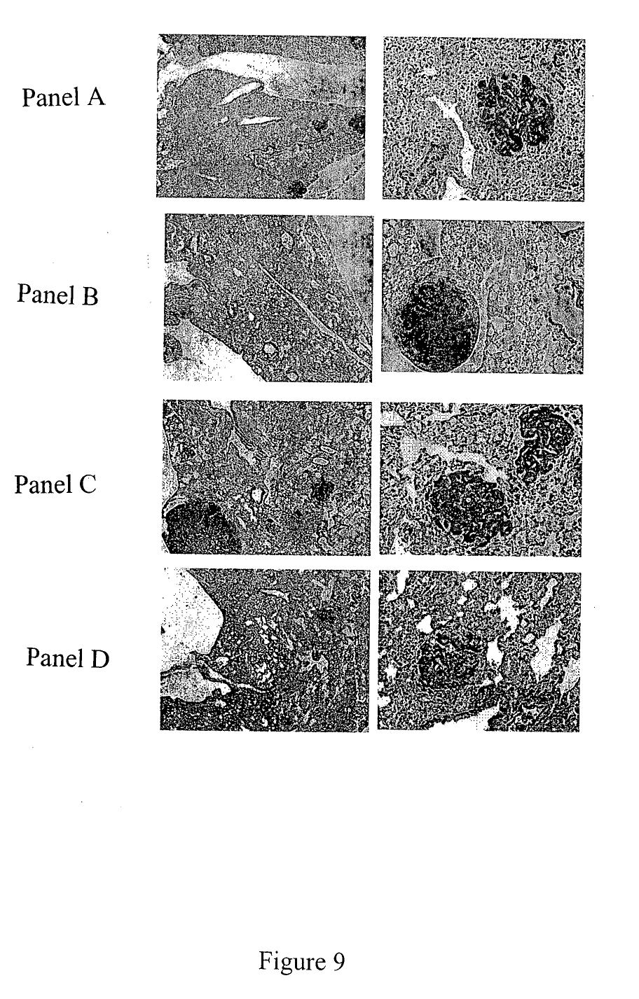

Although the frequency of metastatic foci in the lungs of Akt1 knockout mice is significantly lower (See FIG. 6), the local invasiveness of the tumors developing in all genetic backgrounds is similar. See FIG. 9. However, the histologic appearance of the metastatic foci in both the wild type and the various Akt knockout mice was similar.

### Example V

### Akt does not Regulate Mammary Ductal Architecture

This example shows that Akt specifically regulates mammary tumors, and not non-tumor mammary tissue architecture.

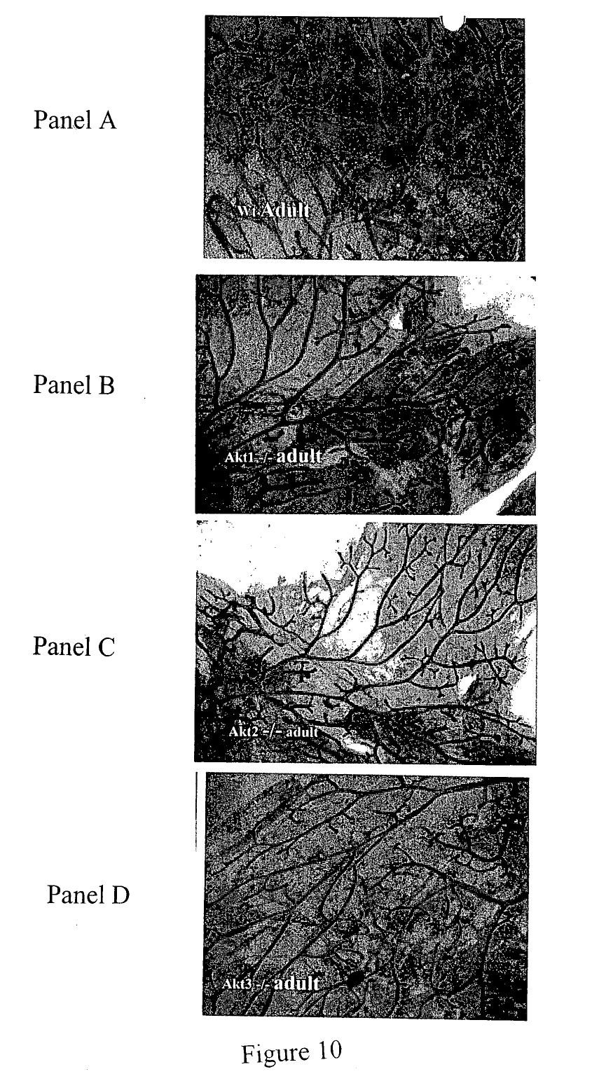

To determine whether Akt1 is required for mammary gland development, mammary gland architecture in young adult (6-8 week old), wild type, Akt1^{-/-}, Akt2^{-/-} and Akt3^{-/-} mice was examined. Analysis of three (3) mice from each group revealed no differences in normal ductal tissue architecture between the groups. See FIG. 10.

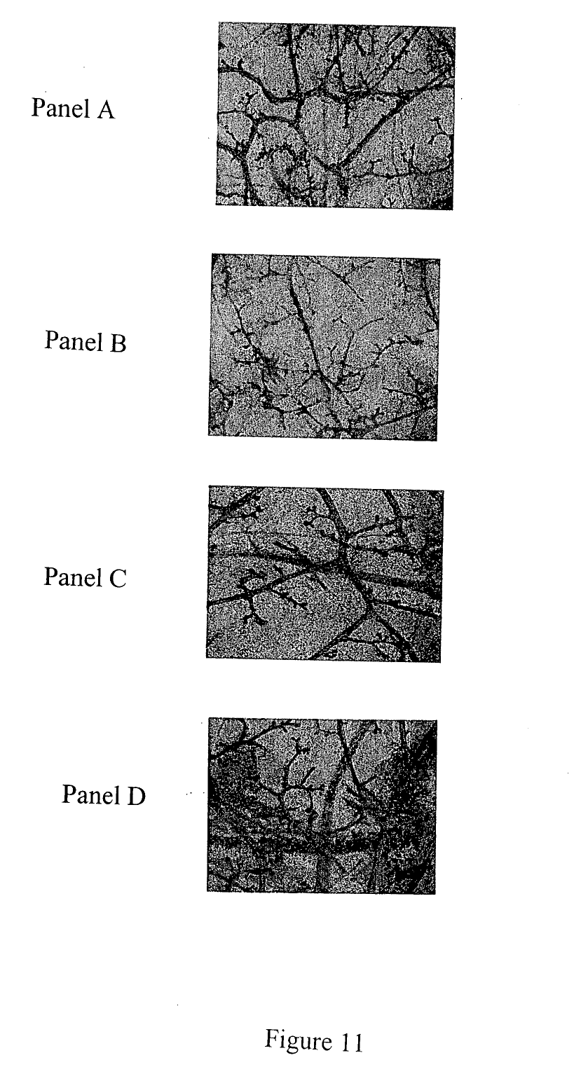

A similar analyses was performed on young MMTV-HER2/Neu or MMTV-PyMT transgenic (10 weeks old and 8 weeks old, respectively), Akt1^{-/-}, Ak12^{-/-} and Akt3^{-/-} and wild type mice. Analysis of three (3) mice from each group revealed again no differences in the branching morphology and the size of normal alveolar tissue outgrowths between wild type and Akt knockout mice carrying either of the two transgenes. See FIG. 11.

Although it is not necessary to understand the mechanism of an invention, it is believed that resistance of mutant mice to the oncogenic potential of transforming genes could be the result of developmental defects that eliminate or limit the number of available target cells.

### Example VI

### Akt1 does not Regulate the MMTV Promoter

This example demonstrates that the ablation of Akt1 does not inhibit the expression of the MMTV promoter-driven transgenes.

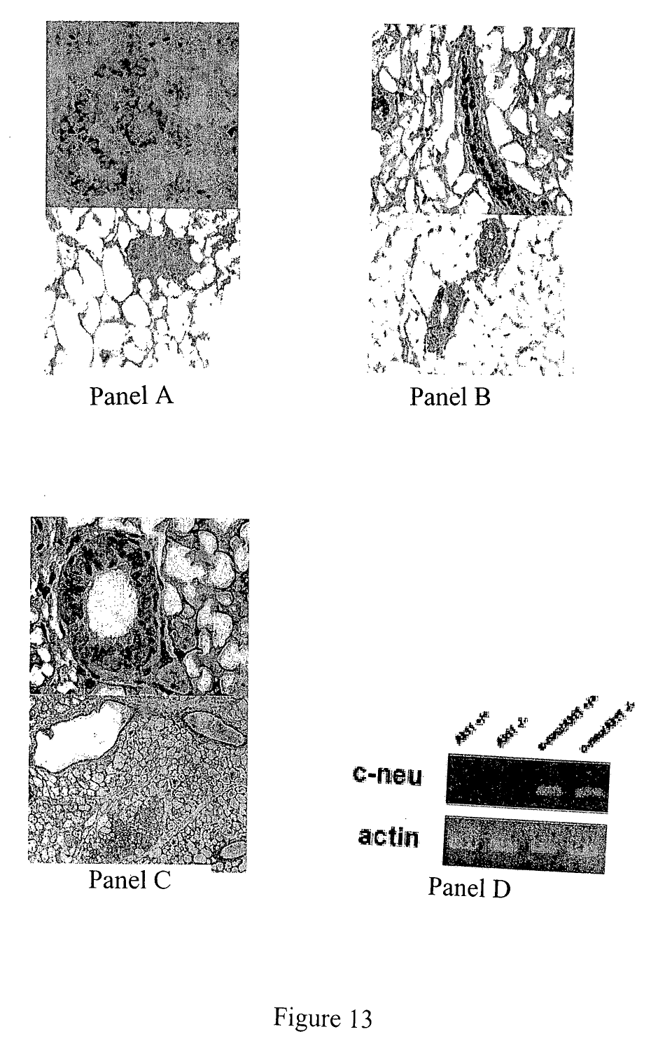

MMTV-HER2/Neu or MMTV-PyMT transgenes were crossed into the Akt1^{-/-}, Akt2^{-/-} and Akt3^{-/-} genetic backgrounds. The induction of the transgenes in 6-8 week old wild type, Akt1, Akt2 and Akt3 knockout mice was examined by immunohistochemistry and semi-quantitative RT-PCR. Immunohistochemistry slides were analyzed for the number of foci expressing the transgene in a given field and for the intensity of staining of individual alveolar epithelial cells. FIG. 13 shows a representative sample of the immunohistochemistry data. These data show that mutations in any of the three Akt isoforms do not affect mammary gland development or the expression of MMTV-driven transgenes.

These data allow the conclusion that the effects of individual Akt isoforms on tumor induction by MMTV-HER2/Neu or MMTV-PyMT, are due to changes in the transduction of HER2/Neu or PyMT-induced oncogenic signals, caused by these mutations.

Although it is not necessary to understand the mechanism of an invention, it is believed that inhibition of these oncogenic signals, due to the ablation of Akt1, was linked to the slow growth of preneoplastic and neoplastic foci, forming in MMTV-PyMT/Akt1^{-/-} mice. It was further hypothesized that the ablation of Akt1 may interfere with tumor induction either by down-regulating the rate of appearance of transformed foci or by inhibiting their rate of growth.

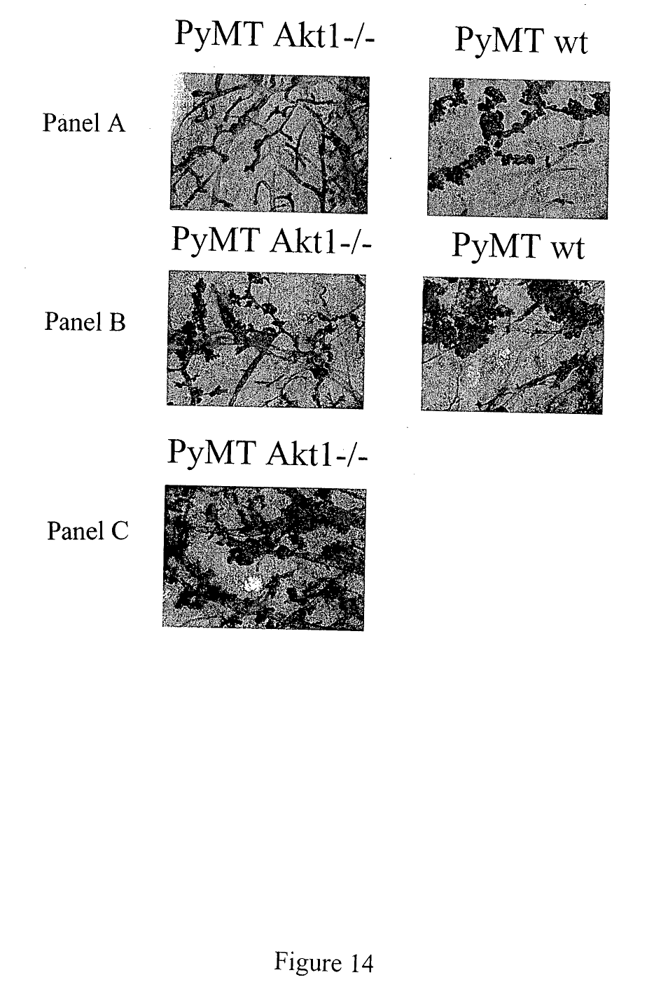

To distinguish between these hypotheses, whole mounds of the mammary glands of 8 week, 12 week and 30 week old MMTV-PyMT/Akt1^{+/+} and MMTV-PyMT/Akt1^{-/-} mice were compared. The comparison involved ten (10) mice from each group. The data revealed that hyperplastic foci can be detected with high frequency in the mammary glands of both the wild type and the Akt1 knockout mice. However, the foci detected in the mammary glands of the Akt1 knockout mice were significantly smaller in all age groups. See FIG. 14. Although it is not necessary to understand the mechanism of an invention, it is believed that these data indicate that the ablation of Akt1 interferes with the transduction of PyMT-generated signals that control the growth of transgene-induced hyperplastic foci.

Consequently, these data support the conclusion that mutations interfering with the induction of a transgene (i.e., for example, an MMTV-driven transgene) that encodes oncogenic proteins should inhibit the oncogenic potential of such transgenes.

### Example VII

### Akt1 Regulates Normal Mammary Epithelia Proliferation

This example shows that PyMT-expressing mammary epithelia from wild type mice exhibit more robust proliferation than Akt1^{-/-} epithelia.

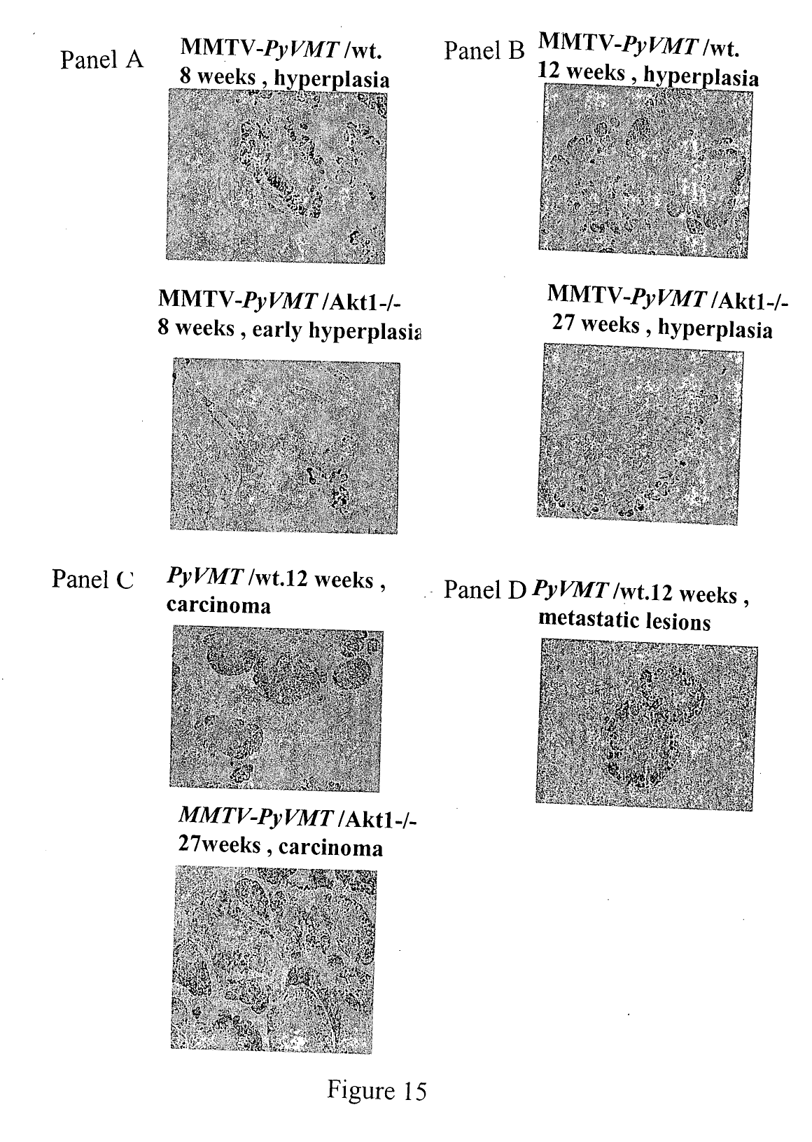

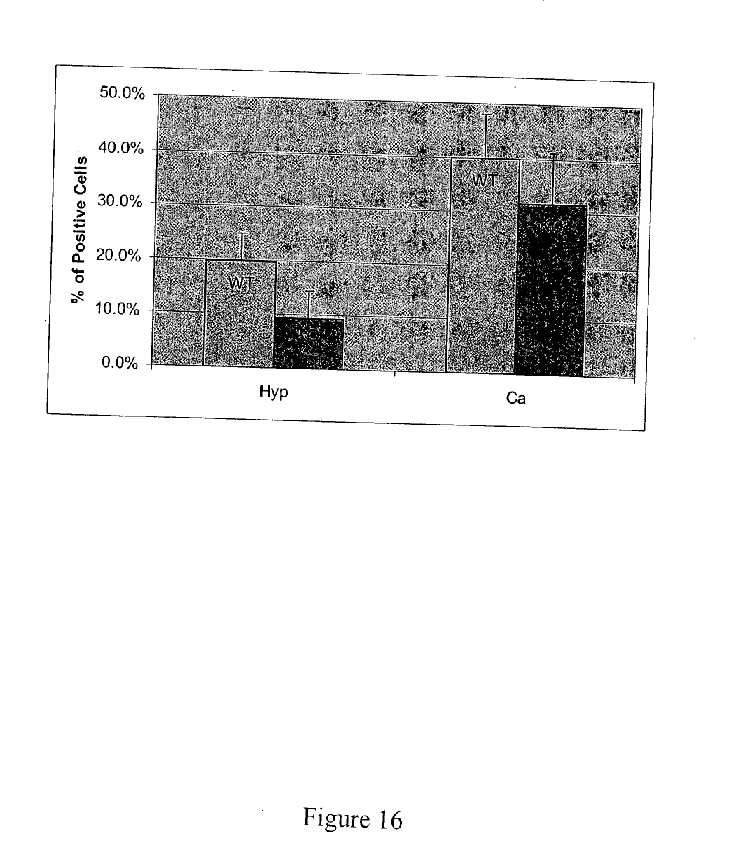

Mammary epithelia of 8 week and 12 week old MMTV-PyMT/Akt1^{+/+} and MMTV-PyMT/Akt1^{-/-} mice were stained with the Ki-67 proliferation marker. Comparison of the mammary epithelia of the Akt1^{+/+} and Akt1^{-/-} mice revealed that the ablation of Akt1 significantly inhibits cell proliferation. See FIG. 15. Ki-67 staining of tumors developing in older Akt1^{-/-} and Akt1^{+/+} mice revealed that tumors differ from the hyperplastic foci in that Akt1^{-/-} and Akt1^{+/+} tumors differ only slightly in their rate of proliferation. See FIG. 16.

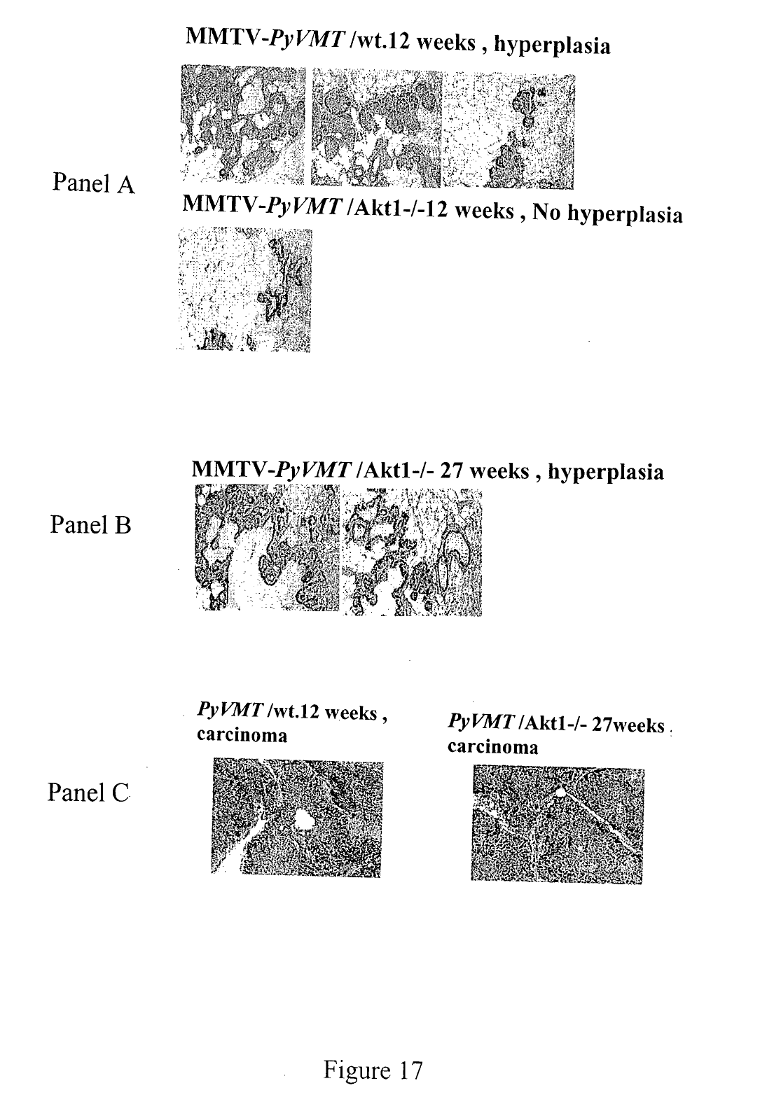

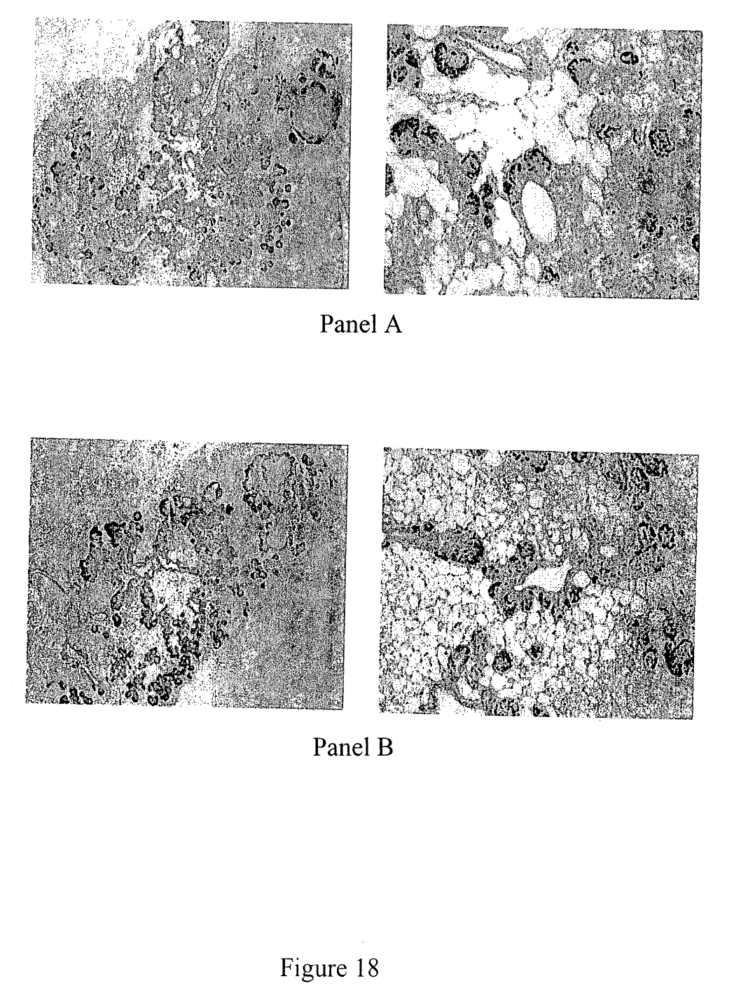

Cyclin D1, a cell cycle regulatory protein whose expression may be regulated by Akt, is believed to play a role in the proliferation of mammary epithelia. Mammary glands from 12 and 27 week old MMTV-PyMT/Akt1^{+/+} and MMTV-PyMT/Akt1^{-/-} mice were stained with an anti-cyclin D1 antibody. See FIG. 17A and FIG. 17B. The results showed that cyclin D1 expression was impaired in the mammary glands of young Akt1^{-/-} mice, harvested prior to tumor induction. Cyclin D1 expression, however, was only slightly higher in tumors developing in older wild type, as opposed to Akt1 knockout mice. See FIG. 17C. Comparison of hyperplastic foci of 6 week old MMTV-PyMT/Akt2^{+/+} and MMTV-PyMT/Akt2^{-/-} mice, for expression of cyclin D1, revealed no differences. See FIG. 18.

### Example VIII

### Akt1 Regulates Mammary Epithelia Apoptosis

This example demonstrates that Akt1 ablation promotes apoptosis in mammary epithelia of MMTV-PyMT transgenic mice.

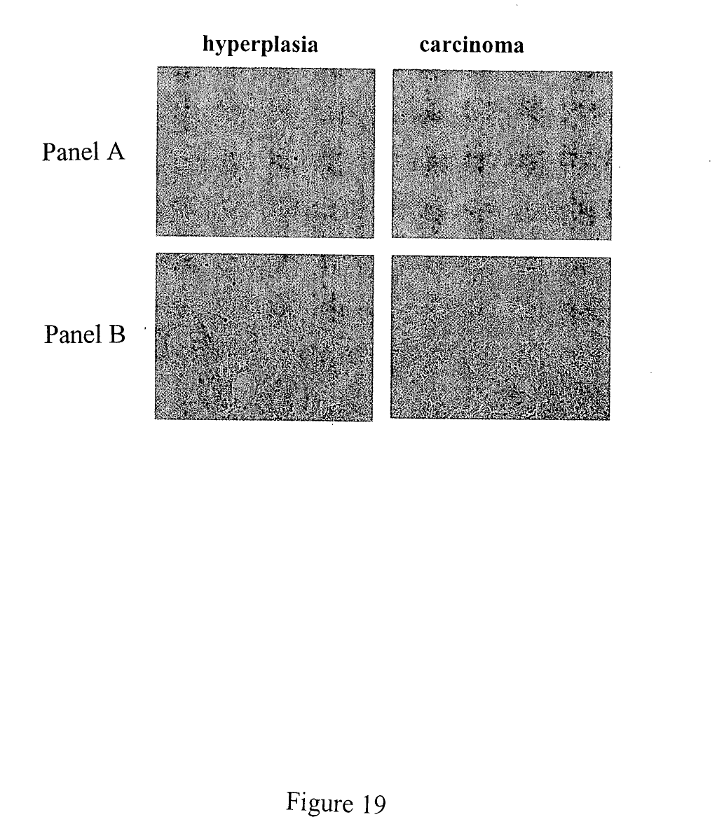

Preneoplastic and neoplastic foci developing in MMTV-PyMT transgenic mice, mammary glands of 8 week old Akt1^{+/+} and Akt1^{-/-} MMTV-PyMT transgenic mice were analyzed for apoptosis, using the TUNNEL assay. This analysis showed approximately twice as many apoptotic cells in the mammary glands of Akt1^{-/-}, as opposed to Akt1^{+/+} mice. See FIG. 19.

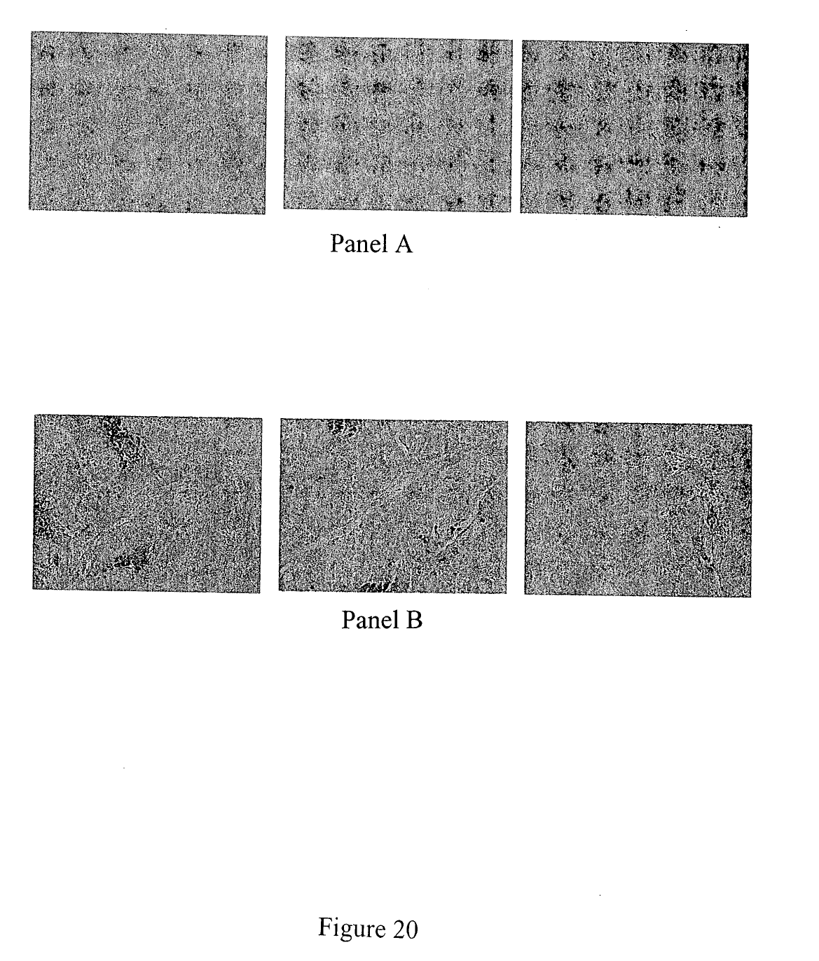

TUNNEL assays were also used to examine the rate of apoptosis in mammary adenocarcinomas developing in Akt1^{+/+} and Akt1^{-/-} MMTV-PyMT mice. This analysis revealed foci of apoptotic cell clusters, which were limited to the Akt1 knockout mice. See FIG. 20. The results of these experiments suggest that ablation of Akt1 impairs the survival of preneoplastic and neoplastic cells. The appearance of the apoptotic cell clusters in Akt1^{-/-} but not in the Akt1^{+/+} tumors, suggested that the rate of growth of established tumors in Akt1^{-/-} mice may be lower than the rate of growth of established tumors in Akt1^{+/+} mice. Furthermore, these observed changes in tumor size increase over time, thereby confirming this prediction (data not shown).

### Example IX

### Effect of Intratumoral Injection of an Akt Gene Product on a Mammary Tumor Lesion

In order to demonstrate the effect of intratumoral injection of an Akt gene product and/or Akt gene product inhibitor, MMTV-PyMT Akt^{-/-} knockout mice (KO mice) will be shaven after the development of a palpable tumor.

Once the tumor reaches a diameter of 4-5 mm the tumors will be injected with either an Akt1 isoform protein and/or an Akt2 isoform inhibitor in 0.1 ml phosphate buffer saline (PBS). The data will show that whereas the control tumors injected with PBS continued to grow many of the tumors injected with an Akt gene product and/or an Akt gene product inhibitor stopped growing and/or displayed a decrease in size (i.e., for example, indicating tumor regression).

### Example X

### Construction of Oncogene/Akt Knockout Mice

MMTV-PyMT and MMTV-HER2/Neu transgenic were purchased from Jackson Laboratories, Bar Harbor Me. Akt1 knockout transgenic mice were constructed by the inventors according to standard techniques. Akt2 and Akt3 knockout transgenic mice were provided by cooperative research groups.

The MMTV-PyMT and/or MMTV-HER2 transgenic mice were crossed with the Akt1, Akt2, and Akt3 transgenic mice to produce the following new transgenic mice:

i) MMTV-PyMT Akt1^{-/-} knockout mice.

ii) MMTV-PyMT Akt2^{-/-} knockout mice.

iii) MMTV-PyMT Akt3^{-/-} knockout mice.

iv) iMMTV-HER2/Neu Akt1^{-/-} knockout mice.

ii) MMTV-HER2/Neu Akt2^{-/-} knockout mice.

iii) MMTV-HER2/Neu Akt3^{-/-} knockout mice.

The appropriate backcrosses were performed to create the representative heterozygotic combinations, and the wild type controls (i.e., Akt^{+/+}).

We claim.:

1. A method, comprising:
a) providing;
i) a patient, said patient comprises a tumor;
ii) a composition comprising an Akt2 isoform protein; and
b) administering said composition to said patient under conditions such that said tumor becomes reduced in size.

2. The method of claim 1, wherein said tumor comprises a mammary tumor.

3. The method of claim 1, wherein said tumor comprises a thymic tumor.

4. The method of claim 1, wherein said protein is a fusion protein.

5. The method of claim 1, wherein said administering is intratumoral.

6. The method of claim 1, wherein said administering is parenteral.

7. A method, comprising:
a) providing;
i) a patient, said patient comprises a tumor;
ii) a composition comprising an Akt1 isoform protein inhibitor; and
b) administering said composition to said patient under conditions such that said tumor becomes reduced in size.

8. The method of claim 7, wherein said tumor comprises a mammary tumor.

9. The method of claim 7, wherein said tumor comprises a thymic tumor.

10. The method of claim 7, wherein said inhibitor is selected from the group consisting of a small molecule and an antibody.

11. The method of claim 7, wherein said administering is intratumoral.

12. The method of claim 7, wherein said administering is parenteral.

13. A transgenic mouse, comprising an MMTV-PyMT transgene operably linked to an Akt1^{-/-} genotype.

14. The transgenic mouse of claim 13, wherein said mouse further comprises a developing tumor.

15. The transgenic mouse of claim 14, wherein said tumor development is inhibited relative to an MMTV-PyMT Akt^{+/+} transgenic mouse.

16. The transgenic mouse of claim 13, wherein said mouse comprises histologically normal tissue selected from the group consisting of mammary ductal architecture and mammary epithelial proliferation.

17. A transgenic mouse, comprising an MMTV-HER2/Neu transgene operably linked to an Akt1^{-/-} genotype.

18. The transgenic mouse of claim 17, wherein said mouse further comprises a developing tumor.

19. The transgenic mouse of claim 18, wherein said tumor development is inhibited relative to an MMTV-PyMT Akt^{+/+} transgenic mouse.

20. The transgenic mouse of claim 17, wherein said mouse comprises histologically normal tissue selected from the group consisting of mammary ductal architecture and mammary epithelial proliferation.

21. A transgenic mouse, comprising an MMTV-PyMT transgene operably linked to an Akt2^{-/-} genotype.

22. The transgenic mouse of claim 21, wherein said mouse further comprises a developing tumor.

23. The transgenic mouse of claim 22, wherein said tumor development is accelerated relative to an MMTV-PyMT Akt^{+/+} transgenic mouse.

24. The transgenic mouse of claim 5, wherein said mouse comprises histologically normal tissue selected from the group consisting of mammary ductal architecture and mammary epithelial proliferation.

25. A transgenic mouse, comprising an MMTV-HER2/Neu transgene operably linked to an Akt2^{-/-} genotype.

26. The transgenic mouse of claim 25, wherein said mouse further comprises a developing tumor.

27. The transgenic mouse of claim 26, wherein said tumor development is accelerated relative to an MMTV-PyMT Akt^{+/+} transgenic mouse.

28. The transgenic mouse of claim 25, wherein said mouse comprises histologically normal tissue selected from the group consisting of mammary ductal architecture and mammary epithelial proliferation.


SEQUENCE LISTING (7 sequences)

SEQ ID NO: 1 - protein, 480 aa, Homo sapiens [sequence omitted]

SEQ ID NO: 2 - DNA, 2878 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 3 - protein, 481 aa, Mus musculus
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRGPGEDAMDYKCGSPSDSSTSEMMEVAVNKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEDRARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLGPEAKSLLAGLLKKDPKQRLGGGPSDAKEVMEHRFFLSINWQDVVQKKLLPPFKPQVTSEVDTRYFDDEFTAQSITITPPDRYDSLDPLELDQRTHFPQFSYSASIRE

SEQ ID NO: 4 - DNA, 3010 bp, Mus musculus
cacgcgtccgcccacgcgtccgctgaaaggagactgtaaaaagtggctctggtgtgtggagctgtggccagggcccctaccactcagctgggagacccaagacgatactgtatccgaggagcctcctgcatgtcctgctgccctgagctcactcaagctaggtgacagcgtgtgaatgctgccaccatgaatgaggtatctgtcatcaaagaaggctggctccacaaacgtggtgaatacatcaagacctggaggccacggtacttccttctgaagagtgatggatctttcattgggtataaggagaggcccgaggcccctgaccagaccttaccccccctgaacaatttctctgtagcagaatgccagctgatgaagactgagaggccacgacccaacacctttgtcatacgctgcctgcagtggaccacagtcatcgagaggaccttccatgtagactctccagatgagagggaagagtggatgcgggctatccagatggtcgccaacagtctgaagcagcggggcccaggtgaggacgccatggattacaagtgtggctcccccagtgactcttccacatctgagatgatggaggtagctgtcaacaaggcacgggccaaagtgaccatgaatgacttcgattatctcaaactcctcggcaagggcaccttcggcaaggtcattctggttcgagagaaggccactggccgctattatgccatgaagatcctgcgcaaggaggtcatcattgcaaaggatgaagtcgcccacacagtcacagagagccgggttctgcagaataccaggcaccccttccttacagccctcaagtatgccttccagacccatgaccgcctatgctttgtgatggagtatgccaacgggggtgagctgtttttccacctctctcgggagcgagtcttcacggaggatcgggcgcgcttttatggagcagagattgtgtcagctctggagtatttgcactcgagagatgtggtgtaccgtgacatcaagctggaaaaccttatgttggacaaagatggccacatcaagatcactgactttggcttgtgcaaagagggcatcagtgatggagccaccatgaaaaccttctgtggtaccccggagtacttggcgcctgaggtgctagaggacaatgactatgggcgagcagtggactggtgggggctgggtgtggtcatgtatgagatgatgtgtggccgcctgccattctacaaccaggaccacgagcgcctctttgagctcattcttatggaggagatccgcttcccgcgcacactcgggccagaggccaagtccctgctggctggactgctgaagaaggacccaaagcagaggctcggcggaggtcccagtgatgcgaaggaggtcatggagcatagattcttcctcagcatcaactggcaggacgtggtacagaaaaagctcctgccacccttcaaacctcaggtcacttcagaagtggacacaaggtactttgatgacgagttcaccgcccagtccatcacaatcacacccccagaccgatatgacagcctggacccgctggaactggaccagcggacgcacttcccccagttctcctactcagccagcatccgagagtgagcagccctctgccaccacaggacacaagcatggccgtcatccactgcctgggtggctttttaaaaaaaaaactttattttgccttttggtttgtgttcccttccctgtcccctcatcccaaatccagtttcttcttcagtccttgctcagacttagtggtcctggcggccctctcctctgggctcccctctgttccaggtgagcgctggggaccgcccagggacaggctgttgggaggatggagagtgaggtcattcctcaccactagcctccaagagggtgaagtggggagccatggggtcacatggatttggaggcacgccagcggaggacacatctagaagttcccctcactcccagcacctcagctgccttccatgctgtcctctgggcaatcttatctgctgccaccccacctggggcacaaaagcagtaatgtccagggacaggcagaagcctttggaagctgaggggtctgggtttgcttctatggagaatggatcttcaagggggatccaggagcaggagctgccttcctcttcctcccaagctactctcttgattctcaataaagcaggaggaatgttcgggggcagccctcctgcccacagcccagtgccttaccagggcttccttctagccagccttgcctcccgctcggcccctttggatggctccgttctctgcctggggcagagggaagccaaccttggctgttacacatctcacaccaagacagtattgggccccttggacttgactggggtctgaggggacagagatggggataggtgtctgatacacaatggggtggagtctgcctcatctccagggcacctgttctggagccttaaagtgaatgtttgagttagagttgtgtcagtgctgtgggtgcgggtgtgcacctgacctagcagtgagcatcccaggctcctgggatgctggtgcccacagctgtatgcagcacttgtgtgagtgggctcaggctcagccccagcctgagtgtttgatcccacggggtgcagtgagtgcaggcgatgtgcctgctgctctggccatgtctctccctcctcaatagctctgggtttttggaaatgagttctcctggttttgctctcccacacacccccagaataggtggatttcagaaaggcagtagggtggggtggttgggatggttatgggggtcatctgttttttactttgtatgtgtgtatgtttatctgagagttttcgcccatccctgctggcctttccttactcctcgtatttgtacggtacaagcaataaagacactcatttcagacccaggaaaaaaaaaaaaaaaaaaaaaaaaaaaaaaaaaaaaaaaaaaaaaaaaaaaaaaaaaaaaaaaaaa

SEQ ID NO: 5 - DNA, 721 bp, Mus musculus
gatatcccagagagtcttggaagtcaccagttagacataacacattcccttcccaggctgattttacctgaggatgtggcgacaaacccattatctggtattaagagtgtgatgcaaacgttccaagagtatccaagataaaacccacccaagactgcaaagaggggtaaagagatgccctgcttttaggaagtgggtgagaactgcaagcaagcaagcaagcgaggcgtcagggcacagcgcgacgcacccagcctgcgccgggagggagaaagtgaagctgggagcagccactcccagtcttgctggaagtcagttggaggggtgggggggcgagccgggagcgcgcggctcccaatcacgggcggcggaggaggcggaggaggagggctgctcgaggaagtgcggcgtgaagttgtggagctgagattgcccgccgctggggacccggagcccaggagcgccccttcccaggcggccccttccggcgccgcgcctgtgcctgccctcgccgcgccccggcccgcagcctggtccagcctgagccatggggccggagccgcagtgatcatcatggagctggcggcctggtgccgttgggggttcctcctcgccctcctgtcccccggagccgcgggtacccaaggtgggtcttggcttggggagggctcgggccgctacgctgcccacggcggccggagccgcggggccccgagagctc

SEQ ID NO: 6 - protein, 367 aa, Mus musculus
MALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPSSPMDSTFYRSLLEDDDMGELVDAEEYLVPQQGFFSPDPALGTGSTAHRRHRSSSARSGGGELTLGLEPSEEEPPRSPLAPSEGAGSDVFDGDLAVGVTKGLQSLSPHDLSPLQRYSEDPTLPLPPETDGYVAPLACSPQPEYVNQPEVRPQSPLTPEGPPPPIRPAGATLERPKTLSPGKNGVVKDVFAFGGAVENPEYLAPRAGTASQPHPSPAFSPAFDNLYYWDQNSSEQGPPPSTFEGTPTAENPEYLGLDVPV

SEQ ID NO: 7 - DNA, 2083 bp, Mus musculus
agggcatctggatcccagatggggagaacgtgaaaatccccgtggccatcaaggtgttgagggaaaacacatctcctaaagctaacaaagaaatcctagatgaagcgtacgtcatggctggtgtgggttctccatatgtgtcccgcctcctgggcatctgcctgacatccacagtgcagctggtgacacagcttatgccctatggctgccttctggaccatgtccgagaacaccgaggtcgcttaggctcccaggacctgctcaactggtgtgttcagattgccaaggggatgagctacctggaggaagttcggcttgttcacagggacctagctgcccgaaacgtgctagtcaagagtcccaaccacgtcaagattaccgacttcgggctggcacggctgctggacattgatgagactgaataccatgcagatgggggcaaggtgcccatcaagtggatggcattggaatctattctcagacgccggttcacccatcagagtgatgtgtggagctatggtgtgactgtgtgggagctgatgacctttggggccaaaccttacgatgggatcccagctcgggagatccctgatttgctggagaagggagaacgcctacctcagcctccaatctgcaccatcgacgtctacatgatcatggtcaaatgttggatgattgactccgaatgtcgcccgagattccgggagttggtatcagaattctcccgtatggcaagggacccccagcgctttgtggtcatccagaacgaggacttaggcccctccagccccatggacagcaccttctaccgttcactgctggaggatgatgacatgggggagctggtcgatgctgaagagtacctggtaccccagcagggattcttctccccagaccctgccctaggtactgggagcacagcccaccgcagacaccgcagctcgtcggccaggagtggcggtggtgagctgacactgggcctggagccctcggaagaagagccccccagatctccactggctccctccgaaggggctggctccgatgtgtttgatggtgacctggcagtgggggtaaccaaaggactgcagagcctctctccacatgacctcagccctctacagcggtacagtgaggatcccacattacctctgccccccgagactgatggctacgttgctcccctggcctgcagcccccagcccgagtatgtgaaccagccagaggttcggcctcagtctcccttgaccccagagggtcctccgcctcccatccgacctgctggtgctactctagaaagacccaagactctctctcctgggaaaaatggggttgtcaaagacgtttttgcctttgggggtgctgtggagaaccctgaatacttagcacccagagcaggcactgcctctcagccccacccttctcctgccttcagcccagcctttgacaacctctattactgggaccagaactcatcggagcagggtcctccaccaagtacctttgaagggacccccactgcagagaaccctgagtacctaggcctggatgtgccagtatgaggtcacatgtgcagacatcctctgtcttcagagtggggaaggaaggcctaacttgtggtctccatcgcccgccacaaagcagggagaaggtcctctggccacatgacatccagggcagccggctatgccaggaacgtgccctgaggaacctcgctcgatgcttcaatcctgagtggttaagagggccccgcctggccggaagagacagcacactgttcagccccagaggattacagaccctgactgccctgacagactgtagggtccagtgggtattccttacctggcctggctctcttggttctgaagactgagggaagctcagcctgcaagggaggaggccccaggtgaatatcctgggagcaggacaccccactaggactgaggcacgtgcatcccaagagggggacagcacttgcacccagactggtctttgtacagagtttattttgttctgtttttacttttgttttttgttttttttttaaagatgaaataaggatacaaaaaaaaaaaaaaaa